(19) United States
(12) Patent Application Publication
(10) Pub. No.: US 2015/0045301 A1
(43) Pub. Date: Feb. 12, 2015

(54) METHODS AND USES OF ANP (ATRIAL NATRIURETIC PEPTIDE), BNP (BRAIN NATRIURETIC PEPTIDE) AND CNP (C-TYPE NATRIURETIC PEPTIDE)-RELATED PEPTIDES AND DERIVATIVES THEREOF FOR TREATMENT OF RETINAL DISORDERS AND DISEASES

(71) Applicant: KALOS Therapeutics, Inc., San Diego, CA (US)

(72) Inventor: Michael Kozlowski, Peoria, AZ (US)

(21) Appl. No.: 14/365,517

(22) PCT Filed: Dec. 17, 2012
(86) PCT No.: PCT/US2012/070190

§ 371 Date: Jun. 13, 2014

Related U.S. Application Data
(60) Provisional application No. 61/576,720, filed on Dec. 16, 2011.

(51) Int. Cl. A61K 38/22; A61K 9/00

(52) U.S. Cl. CPC A61K 38/2242; A61K 9/0048; A61K 38/22
USPC 514/12.4

(57) ABSTRACT

The invention provides NP compounds such as proteins, peptides, peptidomimetics, derivatives and analogs for treating retinal disorders and diseases.

### RELATED APPLICATIONS

This application is the National Phase of International Application No. PCT/US2012/070190, filed Dec. 17, 2012, which designated the U.S. and that International Application was published under PCT Article 21(2) in English, which claims priority to Application Ser. No. 61/576,720, filed Dec. 16, 2011, all of which applications are expressly incorporated herein by reference in their entirety.

### FIELD OF THE INVENTION

The invention relates to proteins, peptides, and peptidomimetics, and derivatives thereof, based upon ANP, BNP and CNP and other structurally related peptides, and methods and uses for treating retinal disorders and diseases.

### INTRODUCTION

It has been observed that naturally occurring peptides, certain medicines, and natural substances can have more than one observed activity. For example, many medicines have side effects, which can appear entirely unrelated and sometimes deleterious to the primary intended effect of drug. However, not all such side effects are deleterious to the health of a patient being dosed with such drugs.

In some cases, observations of an effect of naturally occurring substance indicates that such substances may have some new utility, but the primary observed effect of that substance may overwhelm any observed “new activity” in vivo. As a result such observations remain in vitro, or ex vivo curiosities, since the desired new activity/effect is merely a side effect of the active substance.

One example of such an observation is the finding that Natriuretic Peptides (NPs), which include ANP, BNP, CNP, and urodilatin, in humans, have biological activities in addition to their primary, cardiovascular effects (e.g. the regulation of blood volume, blood pressure, and cardiac function), which include metabolic effects (e.g. control of fat metabolism) and cell growth modulating (e.g. antiproliferative) effects [Potter, et al. Endocrine Rev. 27:47 (2006) and references therein; Vesely, D. L., Curr Heart Fail Rep. 4(3):147 (2007). For example, it has been reported that certain NPs have the ability to kill cancer cells or to attenuate their growth both in culture and in animal models of human cancer [Baldini, et al, Cell Death Diff., 11:S210-S212 (2004); Baldini, et al, Melanoma Res. 16:501-507 (2006); Lelievre, et al., J. Biol. Chem, 276(47):43668, (2001); Levin, et al., Am. J. Physiol. 30:R453-R457 (1991); Vesely, D. L., Eur J Clin Invest 38(8):562 (2008).

In fact, antiproliferative effects have been surprisingly found in NP analogs and peptidomimetics modeled after them that tend to exhibit NPRC-specific activity. Other known molecules [Baldini, et al, Cell Death Diff., 11:S210-S212 (2004); Baldini, et al, Melanoma Res. 16:501 (2006); Gower, et al., Mol. Cell Biochem, 293:103 (2006); Lelievre, et al., J. Biol. Chem, 276(47):43668 (2001); Levin E. R. and Frank, H. J. L., Am. J. Physiol. 30:R453-R457 (1991)] correlate well with this discovery. Moreover, NPRC stimulation modulates the MAP kinase (MAPK) pathway [Hashim, S., Li, Y, Anand-Srivastava, M. B., Am. J. Heart Cir. Physiol, 291:H3144-H3153 (2006); Lelievre, et al., J. Biol. Chem, 276(47):43668 (2001)]. Other types of MAPK inhibitors have been reported to possess anticancer activity [Dhillon, et al., Oncogen 26:3279 (2007)].

Investigators have noted that NP's share common structural features which may contribute to both their receptor binding characteristics and biological activities [He, X-L. et al, Science 293:1657 (2001); Moffatt, et al, J. Biol. Chem. 282:36454 (2007); Potter et al. Endocrine Rev. 27:47 (2006)]. These include a ring structure and a shared amino acid sequence, or motif. Different versions of this motif which have been proposed include CFGXXXDRIXXXXGLGC [Potter, et al., Endocrine Rev. 27:47 (2006)] and CFGXXXDRIXXXXGLGCS [He, et al, Science 293:1657 (2001)]. In each case, the two C residues are connected by a disulfide bridge. An NP motif consisting of FGXX(L/M)DRI(G/S) has also been proposed [Moffatt, et al, J. Biol. Chem. 282:36454 (2007)]. This motif is also found in other endogenous peptides, such as osteocrine and musclin (with replacement of the I residue with an L residue in the latter case), which share some of the metabolic and cell growth modulating effects of the NP's but lack their cardiovascular effects [Moffatt, et al, J. Biol. Chem. 282:36454 (2007); Nishizawa, H., et al., J. Biol. Chem. 279:19391 (2004); Potter, et al., Endocrine Rev. 27:47 (2006) and references therein]. This motif has been termed the Natriuretic Peptide Motif (NM) [Moffatt, et al., J. Biol. Chem. 282:36454 (2007)]. The ring structure of NP's appears necessary for binding to NPRA and NPRB, and for cardiovascular activity [Collinson, P. O., Bus. Brief. Eur. Cardiol, 66 (2005) and references therein; Potter, et al. Endocrine Rev. 27:47 (2006)], and the R14 residue (numbered according to ANP), which is an element of all proposed motifs, is believed to be necessary for binding to NPRC [He, et al, Science 293:1657 (2001); Lanctot, et al, Patent US2007/0049251 A1; Moffatt, et al, J. Biol. Chem. 282:36454 (2007)].

In the case of the NPs, three specific types of NP receptors (NPR) are believed to mediate the actions of NPs. The A-type receptor (NPRA) and the B-type receptor (NPRB) are thought to be involved in NP signaling since they have integral guanylyl cyclase domains [Potter, et al., Endocrine Rev. 27:47 (2006)]. The C-type receptor (NPRC), which has no guanylyl cyclase domain, is thought to be primarily a clearance receptor (i.e. deactivating NP's by reuptake) but it has also been suggested that it is linked to several intracellular signaling pathways [Gower, et al., Mol. Cell Biochem, 293:103 (2006); Hashim et al. Am. J. Heart Cir. Physiol, 291:H3144-H3153 (2006); Lelievre, et al., J. Biol. Chem., 276:43668 (2001); Panayiotou et al. Proc. Brit. Pharm. Soc, 41:abs 009P (2005); Prins, et al, J. Bio. Chem. 271:14156 (1996); Segawa, et al., Naun.-Schmeid. Arch. Pharmacol. 357:70 (1998)].

Each of the NPs appear to interact with these NP receptors, though their affinities each differ for NPRA, NPRB and NPRC. It has been speculated that the relative anticancer potencies of NP's are related to their binding affinities at NPRs [Vesely, D. L., Eur J Clin Invest 38:562 (2008)].

The growth of new blood vessels in the retina is a common feature of several disorders that can have extremely damaging effects on vision [Alexander, L. J. Primary Care of the Posterior Segment. Third Edition, McGraw Hill, New York, 2002, pp 94-98; Kanski, J. J. Age-related macular degeneration. In: Clinical ophthalmology: a systemic approach; Butterworth, Heinemann, Elsevier, N.Y., 2007]. These disorders include proliferative diabetic retinopathy (PDR), retinal vein occlusion, retinopathy of prematuria, “wet” age-related macular degeneration (AMD), pseudoxanthoma elasticum, optic disc drusen, extreme myopia, and malignant myopic degeneration. In the first three of these disorders, the new vessels are extension of the existing retinal vasculature, in the remainder, they result from the growth of choroidal vessels into the retina (choroidal neovascularization; CNV).

The growth of new retinal vessels from either the retinal or choroidal circulation is stimulated by vascular endothelial growth factor (VEGF) [Ciulla, T. A., Rosenfeld, P. J. Curr Opin Ophthalmol; 20:158 (2009); Spaide, R. Etiology of late-age-related macular disease. In: Age related macular degeneration: A comprehensive textbook. Ilfara III, D. V., Liggett, P. E., Mieler, W. F. et al. (eds) 2006; Lippincott, Williams and Wilkins, Philadelphia, pp 23-39]. The incursion of choroidal vessels into the retina may also require a break in the barrier between the choroid and the sensory retina comprising the inner choroidal membrane and the retinal pigmented epithelial (RPE) cell layer. Weakening of the connections between RPE cells can also be promoted by VEGF [Ablonczy, Z., Crosson, C. E. Exp Eye Res 85:762 (2007)] Inhibitors of VEGF are an approved and effective treatment for the CNV resulting from AMD (“wet” AMD), and early results in the treatment of PDR have shown promise [Ciulla, T. A., Rosenfeld, P. J. Curr Opin Ophthalmol; 20:158 (2009); Simo, R., Hernandez, C. Diabetologia 51:1570 (2008)].

Without resorting or limiting the invention to particular mechanistic rationales, the invention described herein is directed to additional activities/effects of natriuretic peptides (NPs), analogues, other peptides and derivatives thereof. In particular, ANP, BNP and CNP and other structurally relates proteins, peptides, peptidomimetics, and derivatives thereof, are useful for treating retinal disorders and diseases. The activities/effects of NPs can also be produced by NPRC-selective NP analogs and peptidomimetics [Baldini, et al, Cell Death Diff., 11:S210-S212 (2004); Baldini, et al, Melanoma Res. 16:501 (2006); Gower, et al., Mol. Cell Biochem, 293:103 (2006); Lelievre, et al., J. Biol. Chem, 276(47):43668 (2001); Levin E. R. and Frank, H. J. L., Am. J. Physiol. 30:R453-R457 (1991)].

### SUMMARY

The invention provides compounds useful for treatment of retinal disorders and diseases. The compounds herein, which include NPs such as ANP, BNP, CNP proteins, peptides, and peptidomimetics, and derivatives and analogs thereof, based upon ANP, BNP, CNP and other structurally related peptides, and referred to collectively herein as “NP-compounds” or simply “compounds” optionally share a conserved motif common among ANP, BNP, CNP and other structurally related peptides. Such NP-compounds can optionally bind to NPRC, or optionally modulate NPRC activity. Invention compounds that contain the conserved motif but lack other structural elements of NPs or NPRC may be selective for treating retinal diseases and disorders and reduce one or more side effects caused by the other structural elements, such as the occurrence of cardiovascular or other side effects mediated by other NPs or NPRs.

NP-compounds include small peptides containing a motif that possesses anti-cell proliferative activity. This motif confers binding to the C-type ANP receptor, which can also lead to inhibition of ERK activation. Based upon a combination of activities, NP-compounds are expected to be effective in inhibiting activity and/or the effects of VEGF, which in turn will lead to treating retinal disorders and diseases, such as retinopathies related to new blood vessel growth (e.g, angiogenesis). The NP-compounds have an advantage over currently available VEGF inhibitors in regards to route of administration. Current inhibitors are large peptides and, therefore, must be administered by intravitreal injection, which is an invasive procedure that can create serious medical complications. Smaller peptides, such as NP-compounds, could potentially be administered orally or topically, thereby reducing or avoiding complications of intravitreal injection to allow more frequent administration, as well as enabling prophylactic in addition to therapeutic use.

### BRIEF DESCRIPTION OF THE DRAWINGS

FIG. 1 show a weakening of the connections between RPE cells in culture, measured as a decrease in their trans-epithelial electrical resistance (TEER), induced by VEGF. This decrease is attenuated by ANP.

### DETAILED DESCRIPTION

Disclosed herein are proteins, peptides, peptidomimetics, and derivatives thereof, based upon NPs, such as ANP, BNP, CNP and other structurally related peptides (referred to collectively as “NP-compounds” or simply “compounds”), useful for treatment of retinal disorders and diseases. Such NP-compounds include those with a conserved motif that is associated with anti-cell proliferative activity useful for treatment of retinal disorders and diseases. The invention therefore provides NP-compounds, including compounds with the conserved motif, and methods and uses of such compounds including, for example, methods for treating retinal disorders and diseases.

In various embodiments, a NP-compound includes a conserved motif, namely, an eight residue compound, denoted (Res1)-(Res 2)-(Res 3)-(Res 4)-(Res 5)-(Res 6)-(Res 7)-(Res 8) [Formula I], wherein each of the residues are contiguous and are bonded (covalently or non-covalently) to their respective adjacent residue. The backbone of the conserved motif can be a linear or cyclic chain, such as a peptide or peptidomimetic chain, typically an amino acid sequence, which may be comprised of one or more naturally occurring amino acids or non-naturally amino acids (including d- or l-amino acids, alpha, beta or gamma amino acid, etc.). Appended to the conserved motif can be additional moieties and/or residues. Appended to the backbone can be side chains as set forth herein. Thus, (Res1)-(Res 2)-(Res 3)-(Res 4)-(Res 5)-(Res 6)-(Res 7)-(Res 8) can have additional moieties and/or residues (e.g., amino acids) adjacent to any of the residues of the conserved motif, as well as side chains appended to the backbone.

Deletion of a terminal residue does not completely attenuate activity, but rather decreases it significantly. For example, deletion of Res 1 and Res8 of Formula 1 (both termini) is expected to decrease the activity proportionately more, without obliterating it altogether. Thus, a conserved motif also includes compounds, peptides and proteins containing the following conserved motif: (Res 2)-(Res 3)-(Res 4)-(Res 5)-(Res 6)-(Res 7) [Formula 2]; (Res 2)-(Res 3)-(Res 4)-(Res 5)-(Res 6)-(Res 7)-(Res 8) [Formula 3; and (Res 1)-(Res 2)-(Res 3)-(Res 4)-(Res 5)-(Res 6)-(Res 7) [Formula 4].

Each of the residues (Res), referred to herein (as Res 1, Res 2, Res 3, Res 4, Res 5, Res 6, Res 7 and Res 8, respectively) are further defined hereinbelow, with increasing specificity. As will be evident, certain positions of the conserved motif allow for significant variation in structure, whereas others require that they are more particularly defined.

Res 1—is an amino acid or mimetic thereof, wherein the backbone is selected from an ether, ester, ketone, alkyl, alkylene or amide linker of 5 or less atoms, having a side chain selected from hydrophobic, non-polar, and non-ionizable side chain, selected from benzyl, alkyl, alkylene, alkenyl, alkylaryl of less than 12 carbons;

Res 2—is an amino acid or mimetic thereof, wherein the backbone is selected from an ether, ester, ketone, alkyl, alkylene or amide linker of 5 or less atoms, having a side chain which is an amphiphilic side chain, hydrophilic side chain, a zwitterion, Glycine (G), Lysine (K), Arginine (R), glutamine (Q), and Asparagine (N);

Res 3—is an amino acid or mimetic thereof, wherein the backbone is selected from an ether, ester, ketone, alkyl, alkylene or amide linker of 5 or less atoms, having a side chain less than 5 members long, except that any ring may be 5 or 6 members in size, and Glycine (G), Histidine (H) Asparagine (N), Serine (S), Leucine (L), or Alanine (A);

Res 4—is a linker of 5 or less atoms or an amino acid, an ether, ester, ketone, alkyl, alkylene, amide linker, with a side chain comprising from 1 to about 12 carbons and with constiuents thereon selected from amino, hydroxyl, amido, carboxy, aryl, heteroaryl and any naturally occurring amino acid;

Res 5—is an amino acid or mimetic thereof, wherein the backbone is selected from an ether, ester, ketone, alkyl, alkylene or amide linker of 5 or less atoms, having a side chain selected from a hydrophobic, non-polar, non-ionizable, aliphatic, side chain, and Leucine (L), Isoleucine (I), Valine (V), Alanine (A), or Methionine (M);

Res 6—is an amino acid or mimetic thereof, wherein the backbone is selected from an ether, ester, ketone, alkyl, alkylene or amide linker 5 or less atoms, having a polar side chain, or amino acids Serine (S), Aspartic Acid (D), Arginine (R), and Glutamic Acid (E);

Res 7—is an amino acid or mimetic thereof, wherein the backbone is selected from an ether, ester, ketone, alkyl, alkylene or amide linker 5 or less atoms, with a side chain comprising from 1 to about 12 carbons and with constiuents thereon selected from amino, hydroxyl, amido, carboxy, aryl, heteroaryl and any naturally occurring amino acid;

Res 8—is an amino acid or mimetic thereof, wherein the backbone is selected from an ether, ester, ketone, alkyl, alkylene or amide linker of 5 or less atoms, having a hydrophobic, non-polar, and non-ionizable side chain, and amino acids Alanine (A), Leucine (L), Isoleucine (I), Valine (V), having a side chain selected from C1 to C12 alkyl, alkylene, and alkenyl.

The conserved motif of certain NP-compounds, as defined herein comprises a “backbone,” and one or more “side chains” appended to the backbone. The term “backbone,” by way of illustration, is a peptide made up of naturally occurring amino acids with a polyamide backbone made up of alpha amino acids. In nature these tend to be L-alpha-amino acids. Conserved motifs, and residues appended thereto can include one or more D-amino acids, and non(un)-natural amino acids, and these D-analogues, as well as any non(un)-natural amino acid's backbones are included within the definition of backbone. In addition, beta-amino acids, or gamma, delta or epsilon analogues, are also included in the backbone definition. As the motif includes peptidomimetics, other moieties to mimic the polyamide backbone, such mimetics can impart improved biological stability and greater half-life in a biological system (e.g., a subject), for example, due, for example, to increased resistance to enzymatic degradation, increased circulation half life, increased bioavailability, increased efficacy, prolonged duration of effect, etc. When referring to peptidomimetics at one more of the residues, a “backbone” includes, for example, an ether, ester, ketone, alkyl, alkylene or amide linker, preferably of 5 or less atoms, with a “side chain” appended thereto.

The term “side chain” by way of illustration, in a naturally occurring peptide is made up of radical attached to the L-polyamide backbone, the 20 “side chains” referred to in the literature are widely known. In addition, less common side chains do occur in nature, some of which are derivatives of natural side chains—these are included in the definition of “side chain.” Whether the side chain is attached to the backbone in an L- or D-configuration, or whether they are in a specific position in an alpha, beta, gamma, delta, or epsilon amino acid is irrelevant since all are included in the definition of side chain. The term side chain includes the panoply of functionality embodied in naturally occurring amino acids and variations thereof, hence sufhydryl, sulphone, carboxy, animo, amido, hydroxyl, alkyl, alkylaryl (or heteroaryl), aryl or heteroaryl radicals are all included in this definition. For example, exemplary hydrophobic side chains include, but are not limited to benzyl, alkyl, alkylene, alkenyl, alkylaryl of less than 12 carbons. The skilled artisan, in view of the specification understands the breadth and variety as to the term “side chain.” As it relates to peptidomimetics, the backbone, has appended thereto 1-3 radicals, as defined above as side chains. Typically, up to 2 and more typically one side chain exists per residue (e.g., Res 1, Res, 2, . . . etc.)

For purposes of illustrating the conserved motif, oligopeptides using known amino acids, are exemplified not to limit the invention, but to illustrate non-limiting embodiments. For example, an oligopeptide, comprising about eight amino acids, forms a conserved anti-cell proliferative motif. Such motifs may exhibit increased activity by increased length (e.g., 9, 10, 11, 12, 13, 14, 15, 16, 17, 8, 19, 20, etc., or more residues). Of course, peptidomimetics, including unnatural amino acids, derivatives and analogues at one or more positions of the sequence, whether within or outside or appended to the conserved motif, are included.

In particular embodiment, ANP-related peptide compound has a conserved motif denoted as: (Res 1)-(Res 2)-(Res 3)-(Res 4)-(Res 5)-(Res 6)-(Res 7)-(Res 8), wehre each of Res 1 to Res 8 is defined as:

Res 1—a hydrophobic, non-polar, and non-ionizable side chain, illustrated by amino acids including Phenylalanine (F), Alanine (A), Leucine (L), Isoleucine (I), Valine (V). Exemplary side chains include alkyl, alkylene, alkenyl, alkylaryl, and the like, typcially of less than 12 carbons, more typically C1-C5 alkyl, or C1-C3 alkyl with an aryl substituent thereon.

Res 2—is an amphiphilic or hydrophilic side chain, which may include a zwitterion, illustrated by amino acids including Glycine(G), Lysine (K), Arginine (R), glutamine (Q), Asparagine (N).

Res 3 is a amino acid side chain which is small in size, typically a chain less than 5 members long, except that any ring may be 5 or 6 members in size, and more preferably C1 to C3 in length if alkyl, more preferably C0-C2, illustrated by amino acids including Glycine(G), Histidine (H) Asparagine (N), Serine (S), Leucine (L), Alanine (A).

Res 4—is a linker of about the size of an amino acid, with a side chain which may vary C1-C12, and have any constituent, as represented by the diversity found in the 20 naturally occurring amino acids. As a result an ether, ester, ketone, alkyl, alkylene or amide linker, with substituents thereon, of any similar size to an amino acid may be suitable.

Res 5—is a hydrophobic, non-polar, non-ionizable, aliphatic, side chain, as illustrated by amino acids including Leucine (L), Isoleucine (I), Valine (V), Alanine (A), or Methionine (M).

Res 6—is a polar side chain, illustrated by amino acids Serine (S), Aspartic Acid (D), Arginine (R), and Glutamic Acid (E).

Res 7—is a linker of about the size of an amino acid, with a side chain which may vary C 1-C12, and have any constituent, as represented by the diversity found in the 20 naturally occurring amino acids. As a result an ether, ester, ketone, alkyl, alkylene or amide linker, with substituents thereon, of any similar size to an amino acid may be suitable.

Res 8—is a hydrophobic, non-polar, and non-ionizable side chain, illustrated by amino acids including, Alanine (A), Leucine (L), Isoleucine (I), Valine (V). Exemplary side chains will be alkyl, alkylene, alkenyl, and the like, typically of less than 12 carbons, more typically C1-C5 alkyl, or C1-C3 alkyl with an aryl substituent thereon.

Suitable side chains may be selected based on size, polarity, acidity or basicity, and hydrophobicity using the known structures, and known parameters such as those selected and illustrated below:

| | | | | | | | | van der |
|---|---|---|---|---|---|---|---|---|
| AA 1 | | Hydro- | | | | | Aromatic | Waals |
| letter | Side chain | phobic | pKa | Polar | pI | pH | or Aliphatic | volume |
| A | —CH₃ | X | — | — | 6.01 | — | — | 67 |
| C | —CH₂SH | X | 8.18 | — | 5.05 | acidic | — | 86 |
| D | —CH₂COOH | — | 3.90 | X | 2.85 | acidic | — | 91 |
| E | —CH₂CH₂COOH | — | 4.07 | X | 3.15 | acidic | — | 109 |
| F | —CH₂C₆H₅ | X | — | — | 5.49 | — | Aromatic | 135 |
| G | —H | — | — | — | 6.06 | — | — | 48 |
| H | —CH₂—C₃H₃N₂ | — | 6.04 | X | 7.60 | weak | Aromatic | 118 |
| | | | | | | basic | | |
| I | —CH(CH₃)CH₂CH₃ | X | — | — | 6.05 | — | Aliphatic | 124 |
| K | —(CH₂)₄NH₂ | — | 10.54 | X | 9.60 | basic | — | 135 |
| L | —CH₂CH(CH₃)₂ | X | — | — | 6.01 | — | Aliphatic | 124 |
| M | —CH₂CH₂SCH₃ | X | — | — | 5.74 | — | — | 124 |
| N | —CH₂CONH₂ | — | — | X | 5.41 | — | — | 96 |
| P | —CH₂CH₂CH₂— | X | — | — | 6.30 | — | — | 90 |
| Q | —CH₂CH₂CONH₂ | — | — | X | 5.65 | — | — | 114 |
| R | —(CH₂)₃NH— | — | 12.48 | X | 10.76 | strongly | — | 148 |
| | C(NH)NH₂ | | | | | basic | | |
| S | —CH₂OH | — | — | X | 5.68 | — | — | 73 |
| T | —CH(OH)CH₃ | — | — | X | 5.60 | weak | — | 93 |
| | | | | | | acidic | | |
| U | —CH₂SeH | X | 5.73 | — | | — | — | |
| V | —CH(CH₃)₂ | X | — | — | 6.00 | — | Aliphatic | 105 |
| W | —CH₂C₈H₆N | X | — | — | 5.89 | — | Aromatic | 163 |
| Y | —CH₂—C₆H₄OH | — | 10.46 | X | 5.64 | — | Aromatic | 141 |

In addition, substitution of one or more amino acids, alterations of the backbone with derivatives, analogs (peptidomimetics) and other alterations as described herein are also contemplated.

As disclosed herein, invention NP-compounds that include a conserved motif can include additional moieties and/or residues (e g , amino acids or mimetics thereof) adjacent to any of the residues of the conserved motif (e.g., any one of (Res1)-(Res 2)-(Res 3)-(Res 4)-(Res 5)-(Res 6)-(Res 7)-(Res 8)). Typically, the number of residues in an invention compound will total less than about 150 residues (e g , amino acids or mimetics thereof), inclusive of a conserved motif. In various particular embodiments, the number of residues comprise from about 8 up to about 140 residues (e.g., amino acids or mimetics thereof). In additional embodiments, the number of residues comprise from about 8 up to about 125 residues (e.g., amino acids or mimetics thereof). In further embodiments, the number of residues comprise from about 8 up to about 100 residues (e g , amino acids or mimetics thereof). In yet additional embodiments, the number of residues comprise from about 8 up to about 90 residues (e.g., amino acids or mimetics thereof), or between about 10 to 80, 10 to 70, 10 to 60, 10 to 50, 10 to 40, 10 to 30, 10 to 25, or 10 to 20 residues (e.g., amino acids or mimetics thereof) in length. In particular aspects.

While not intending to be bound by any particular theory or mechanism, it appears that binding to the NPRC receptor can be predictive of activity. Thus, although compounds of the invention include species with and without the motif, and compounds having the conserved motif that are incapable of binding the NPRC receptor, typically compounds having the conserved motif are capable of binding the NPRC receptor, optionally selectively, and more typically are capable of binding the NPRC receptor with an equilibrium receptor binding affinity to the NPRC receptor, determined by the Ki value, in at least the micromolar range. Furthermore, it appears that compounds that the conserved motif do not require the C residues for a ring structure, nor the R14 residue that has been deemed critical for activity in all previously identified motifs.

The conserved motif is shared by ANP, CNP, and BNP, and other related peptides such as LANP, KP, VDL, and the ANP analog, cANF, all of which have activity. CNP, which is less active than ANP, LANP, KP or VDL has a slightly larger amino acid side chain on one of the residues of the motif (L instead of G, A, N or S). BNP, has an even larger side chain at this position (K), and one which differs in electronic characteristics. Small molecule ANP derivatives, which also bind to NPRC, also have structures which are explainable based on the motif. For example, elimination of the equivalent of the R14 residue in one these molecules did not eliminate activity. Accordingly, all of such proteins, peptides, mimetics, derivatives and analogs are considered to be within the class of NP-compounds set forth herein. Since the conserved motif is significantly different from previously described NP motifs, and NP motifs are believed to underlie the cardiovascular effects of these peptides, compounds that contain the conserved motif without the NP motif(s) are predicted to have desired activity with fewer if any cardiovascular side effects of native NP peptides or proteins and are therefore included.

The conserved motif “residues” (i.e., amino acids or mimetics thereof) as well as their subunits, in this invention can be a) naturally or non-naturally occurring, b) produced by chemical synthesis, c) produced by recombinant DNA technology, d) produced by, chemical, biochemical or enzymatic fragmentation of larger molecules, e) produced by methods resulting from a combination of methods a through d listed above, or f) produced by any other means for producing peptides or amino acid sequences. By employing chemical synthesis, it is possible to introduce various amino acids which do not naturally occur into the construct, modify the N- or C-terminus, and the like, thereby providing improved stability (on the shelf or in vivo) and formulation, resistance to protease degradation, etc., and to introduce one or more amino acid surrogates into the construct.

The term “peptide” as used herein includes any structure comprised of two or more amino acids, including chemical modifications and derivatives of amino acids, covalently linked. The amino acids forming all or a part of a peptide may be naturally occurring amino acids, stereoisomers and modifications of such amino acids, non-protein amino acids, post-translationally modified amino acids (e.g., by glycosylation, ester or amide cleavage, etc.), enzymatically modified amino acids, amino acids with their side chain moiety either modified, derivatized from naturally occurring moieties, or are entirely synthetic, or not naturally occurring. The term “peptide” also includes dimers or multimers of peptides. A “manufactured” peptide includes a peptide produced by chemical synthesis, recombinant DNA technology, biochemical or enzymatic fragmentation of larger molecules, combinations of the foregoing or, in general, made by any other method involving manipulation by the hand of man.

The term “amino acid side chain moiety” as used herein includes any side chain of any amino acid, as the term “amino acid” is defined herein. This therefore includes the side chain moiety present in naturally occurring amino acids. It further includes side chain moieties in modified naturally occurring amino acids, such as side chain moieties in stereoisomers and modifications of naturally occurring protein amino acids, non-protein amino acids, post-translationally modified amino acids, enzymatically synthesized amino acids, derivatized amino acids, constructs or structures designed to mimic amino acids, etc. For example, the side chain moiety of any amino acid disclosed herein or known to one of skill in the art is included within the definition. A “derivative of an amino acid side chain moiety” is included within the definition of an amino acid side chain moiety.

The skilled artisan will recognize that amino acid side chain moieties can be derivatized, including a modification to or variation in either a naturally occurring or unnatural amino acid side chain moiety, wherein the modification or variation includes, for example, but is not limited to: (a) adding one or more saturated or unsaturated carbon atoms to an existing alkyl, aryl, or aralkyl chain; (b) substituting a carbon in the side chain with another atom, preferably oxygen or nitrogen; (c) adding a terminal group to a carbon atom of the side chain, including methyl (—CH3), methoxy (—OCH3), nitro (—NO2), hydroxyl (—OH), or cyano (—C═N); (d) for side chain moieties including a hydroxy, thiol or amino groups, adding a suitable hydroxy, thiol or amino protecting group; or (e) for side chain moieties including a ring structure, adding one or ring substituents, including hydroxyl, halogen, alkyl, or aryl groups attached directly or through an ether linkage. For amino groups, suitable protecting groups are known to the skilled artisan. Provided such derivatization retains at least partial activity in the final compound, all such derivatization is included in the definition of “amino acid side chain moiety.”

The “amino acids” herein include naturally and non-naturally occurring amino acids (residues), such as L- and D-forms, respectively, as well as derivatives and analogs thereof. Naturally occurring protein amino acids are referred to by both their common three-letter abbreviation and single letter abbreviation (see generally Synthetic Peptides: A User's Guide, G. A. Grant, editor, W.H. Freeman & Co., New York (1992), which is incorporated herein by reference). An “amino acid” includes conventional alpha-amino acids as well as beta-amino acids, alpha, alpha disubstituted amino acids and N-substituted amino acids wherein at least one side chain is an amino acid side chain moiety as defined herein. An “amino acid” further includes N-alkyl alpha-amino acids, wherein the N-terminus amino group has a C1 to C6 linear or branched alkyl substituent. The term “amino acid” therefore includes derivatives (e.g., stereoisomers and modifications) of naturally occurring protein amino acids, non-protein amino acids, post-translationally modified amino acids, enzymatically synthesized amino acids, derivatized amino acids, constructs or structures designed to mimic amino acids, etc. Modified and unusual amino acids are included in the compounds of the invention and are described generally, for example, in Synthetic Peptides: A User's Guide, cited above; Hruby V. J., Al-obeidi F., Kazmierski W., Biochem. J. 268:249-262 (1990); and Toniolo C., Int. J. Peptide Protein Res. 35:287 (1990).

In addition, the following amino acids and protecting and modifying groups thereof, are included: gamma-amino butyric acid, 12-amino dodecanoic acid, alpha-aminoisobutyric acid, 6-amino hexanoic acid, 4-(aminomethyl)-cyclohexane carboxylic acid, 8-amino octanoic acid, biphenylalanine, Boc-t-butoxycarbonyl, benzyl, benzoyl, citrulline, diaminobutyric acid, pyrrollysine, diaminopropionic acid, 3,3-diphenylalanine, orthonine, citrulline, 1,3-dihydro-2H-isoindolecarboxylic acid, ethyl, Fmoc-fluorenylmethoxycarbonyl, heptanoyl (CH3-(CH2). sub. 5-C(═)—), hexanoyl (CH3-(CH2)4-C(═O)—), homoarginine, homocysteine, homolysine, homophenylalanine, homoserine, methyl, methionine sulfoxide, methionine sulfone, norvaline (NVA), phenylglycine, propyl, isopropyl, sarcosine (SAR), tert-butylalanine, benzyloxycarbonyl

In the compounds of the invention, conventional amino acid residues have their conventional meaning. Thus, “Nle” is norleucine, and so on. While it is expected that either D or L residues are active and are included in the invention, for the purposes of the examples, and to save space and time in the Sequence Listing information, residues listed in examples are in the L-isomer configuration unless the D-isomer is specified, for example, as in “D-Ala” or “D-A” for D-Alanine. Nevertheless, D-amino acids at any or all positions of the compounds of the invention are included.

A single amino acid, including stereoisomers and modifications of naturally occurring protein amino acids, non-protein amino acids, post-translationally modified amino acids, enzymatically synthesized amino acids, non-naturally occurring amino acids including derivatized amino acids, an alpha, alpha disubstituted amino acid derived from any of the foregoing (i.e., an alpha, alpha disubstituted amino acid, wherein at least one side chain is the same as that of the residue from which it is derived), a beta-amino acid derived from any of the foregoing (i.e., a beta-amino acid which other than for the presence of a beta-carbon is otherwise the same as the residue from which it is derived) etc., including all of the foregoing can be referred to herein as a “residue.” Suitable substituents, in addition to the side chain moiety of the alpha-amino acid, include C1 to C6 linear or branched alkyl. Aib is an example of an alpha, alpha disubstituted amino acid. While alpha, alpha disubstituted amino acids can be referred to using conventional L- and D-isomeric references, it is to be understood that such references are for convenience, and that where the substituents at the alpha-position are different, such amino acid can interchangeably be referred to as an alpha, alpha disubstituted amino acid derived from the L- or D-isomer, as appropriate, of a residue with the designated amino acid side chain moiety. Thus (S)-2-Amino-2-methyl-hexanoic acid can be referred to as either an alpha, alpha disubstituted amino acid derived from L-Nle or as an alpha, alpha disubstituted amino acid derived from D-Ala. Similarly, Aib can be referred to as an alpha, alpha disubstituted amino acid derived from Ala. Whenever an alpha, alpha disubstituted amino acid is provided, it is to be understood as including all (R) and (S) configurations thereof.

An “N-substituted amino acid” includes any amino acid wherein an amino acid side chain moiety is covalently bonded to the backbone amino group, optionally where there are no substituents other than H in the alpha-carbon position. Sarcosine is an example of an N-substituted amino acid. By way of example, sarcosine can be referred to as an N-substituted amino acid derivative of Ala, in that the amino acid side chain moiety of sarcosine and Ala is the same, methyl.

The term “any amino acid” as used herein refers to any such naturally or non-naturally occurring amino acids, such as L- and D-forms, as well as derivatives, mimetics, modified forms, and analogs thereof. Thus, by way of example, “any amino acid” includes any one of the following naturally occurring residues, such as histidine, arginine, phenylalanine, alanine, cysteine, glycince, glutamine, aspartic acid, glutamic acid, lyseine, leucine, methionine, asparagine, serine, tyrosine, threonine, tryptophan, isoleucine, proline, and valine, as well as any non-natural equivalent, as well as derivatives, mimetics, modified forms, and analogs thereof.

The term “peptidomimetic” includes a molecule disclosed herein which is a mimic of a residue (referred to as a “mimetic”), including but not limited to piperazine core molecules, keto-piperazine core molecules and diazepine core molecules. Unless otherwise specified, an amino acid mimetic of an invention compound includes both a carboxyl group and amino group, and a group corresponding to an amino acid side chain, or in the case of a mimetic of Glycine, no side chain other than hydrogen.

By way of example, these would include compounds which mimic the sterics, surface charge distribution, polarity, etc. of a naturally occurring amino acid, but need not be an amino acid, which would impart stability in the biological system. For example, Proline may be substituted by other lactams or lactones of suitable size and substitution; Leucine may be substituted by an alkyl ketone, N-substituted amide, as well as variations in amino acid side chain length using alkyl, alkenyl or other substituents, others may be apparent to the skilled artisan. The essential element of making such substitutions is to provide a molecule of roughly the same size and charge and configuration as the residue used to design the molecule. Refinement of these modifications will be made by testing the compounds in a binding or other assay, and analysis of the structure activity relationship. Such methods are within the scope of the skilled artisan working in medicinal chemistry and drug development.

NP-compounds of the invention can have more than one asymmetric center, and are therefore capable of existing in more than one stereoisomeric form. Some of the compounds may also exist as geometric isomers and rotamers. Furthermore, some compounds of the invention may also have conformational axial chirality. The invention extends to each of these forms individually and to mixtures thereof, including racemates. In one aspect, isomers may be separated conventionally by chromatographic methods or by use of a resolving agent. In another aspect, individual isomers, or enantiomerically pure isomers, are prepared by synthetic schemes, such as those disclosed herein or variants of such schemes, employing asymmetric synthesis using chiral intermediates, reagents or catalysts.

Either terminus of the conserved motif, or any peptide comprising the conserved motif, may be protected from degradation by any terminal group. For example, at the C-terminus a terminal group attached through the terminal carbon atom or, if provided, terminal carboxyl group, of the C-terminus of a construct attached thereto. The terminal ring carbon atom or, if provided, terminal carboxyl group, may form a part of a residue, or may form a part of an amino acid surrogate. For example, the C-terminus capping group forms a part of an amino acid surrogate which is at the C-terminus position of the construct. The C-terminus capping group includes, but is not limited to, —(CH2)n-OH, —(CH2)n-C(═O)—OH, —(CH2)m-OH, —(CH2)n-C(═O)—N(V1)(V2), —(CH2)n-C(═O)—(CH2)m-N(V1)(V2), —(CH2)n-O—(CH2)m-CH3, —(CH2)n-C(═O)—NH—(CH2)m-CH3, —CH2)n-C(═O)—NH—(CH2)m-N(V1)(V2), —(CH2)n-C(═O)—N—((CH2)m-N(V1)(V2))-2, —(CH2)n-C(═O)—NH—CH(—C(═O)—OH)—(CH2)-m-N(V1)(V2), —C(═O)—NH—(CH2)m-NH—C(═O)—CH(N(V1)(V2))-((CH2)m-N(V1)(V2)), or —(CH2)n-C(═O)—NH—CH(—C(═O)—NH2)-(CH2).-m-N(V1)(V2), including all (R) or (S) configurations of the foregoing, where V1 and V2 are each independently H, a C1 to C7 linear or branched alkyl chain, m is 0 to 7 and n is 0 to 2; or any omega amino aliphatic, terminal aryl or aralkyl, including groups such as methyl, dimethyl, ethyl, propyl, isopropyl, butyl, isobutyl, pentyl, hexyl, allyl, cyclopropane methyl, hexanoyl, heptanoyl, acetyl, propionoyl, butanoyl, phenylacetyl, cyclohexylacetyl, naphthylacetyl, cinnamoyl, phenyl, benzyl, benzoyl, 7′-amino heptanoyl, other groups known in the art, or any single natural or unnatural alpha-amino acid, beta-amino acid or alpha, alpha disubstituted amino acid, including all chiral configurations of the foregoing, optionally in combination with any of the foregoing non-amino acid capping groups.

The N-terminus of a NP-compound including a conserved motif, or a compound comprising the motif, may be protected from degradation by any terminal group attached through the terminal group attached through the terminal amine of the N-terminus of a construct. The terminal amine may form a part of a residue, or may form a part of an amino acid surrogate. For example, the N-terminus capping group forms a part of an amino acid surrogate, which is at the N-terminus position of the construct. The N-terminus capping group includes, but is not limited to, any omega amino aliphatic, acyl group or terminal aryl or aralkyl including groups such as methyl, dimethyl, ethyl, propyl, isopropyl, butyl, isobutyl, pentyl, hexyl, allyl, cyclopropane methyl, hexanoyl, heptanoyl, acetyl, propionoyl, butanoyl, phenylacetyl, cyclohexylacetyl, naphthylacetyl, cinnamoyl, phenyl, benzyl, benzoyl, 7′-amino heptanoyl, other groups knwn in the art, or alternatively an N-terminus capping group is —(CH2)m-NH(V3), —(CH2)m-CH3, —C(═O)—(CH2)m-CH3, —C(═O)—(CH2)m-NH(V3), ——C(═O)—(CH2)m-C(═O)—OH, —C(═O)—(CH2)m-C(═O)—(V4), —(CH2)m-C(═O)—OH, —(CH2)m-C(═O)—(V4), C(═O)—(CH2)m-O(V3), —(CH2)m-O(V3), C(═O)—(CH2)m-S(V3), or —(CH2)m-S(V3), where V3 is H or a C1 to C17 linear or branched alkyl chain, and V4 is a C 1 to C17 linear or branched alkyl chain and m is 0 to 17. A phenyl ring is “substituted” when the phenyl ring includes one or more substituents independently comprising hydroxyl, halogen, alkyl, or aryl groups attached directly or through an ether linkage. Where the phenyl ring is so substituted, the amino acid residue may be referred to as substituted, as in substituted Phe, substituted HPhe.

The variations can be made using methods known to one skilled in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] and other techniques can be performed on the cloned DNA to produce invention compounds or variations, derivatives, substitutions or modifications thereof.

Covalent modifications of the compound are included within the invention. One type of covalent modification includes reacting targeted amino acid residues with an organic derivatizing agent that is capable of reacting with selected side chains or the N- or C-terminal residues of the peptide. Derivatization with bifunctional agents is useful, for instance, for cross linking peptide to a water-insoluble support matrix or surface for use in the method for purifying anti-peptide antibodies, and vice-versa. Commonly used cross linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the alpha-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, amidation of any C-terminal carboxyl group, etc.

Another type of covalent modification of the one or more residues of the compound of the invention includes glycosylation. “Glycosylation” is intended to mean adding or deleting one or more carbohydrate moieties (either by removing the underlying glycosylation site or by deleting the glycosylation by chemical and/or enzymatic means), and/or adding one or more glycosylation sites that may or may not be present in the native sequence. In addition, the phrase includes qualitative changes in the glycosylation of the native proteins, involving a change in the nature and proportions of the various carbohydrate moieties present.

Addition of glycosylation sites to the compound can be accomplished by altering the amino acid sequence. The alteration may be made, for example, by the addition of, or substitution by, one or more serine or threonine residues (for O-linked glycosylation sites) or asparagine (N-linked glycosylation site) to the compound. Of course, the compound may optionally be altered through changes at the DNA level, particularly by mutating the DNA encoding the peptide polypeptide at preselected bases such that codons are generated that will translate into the desired amino acids. Another means of increasing the number of carbohydrate moieties on the peptide polypeptide is by chemical or enzymatic coupling of glycosides to the polypeptide (see, for example, in WO 87/05330; and in Aplin and Wriston, CRC Crit. Rev. Biochem., pp. 259 (1981)). Removal of carbohydrate moieties may be accomplished chemically or enzymatically or by mutational substitution of codons encoding for amino acid residues that serve as targets for glycosylation. Chemical deglycosylation techniques are known (see, for example, Hakimuddin, et al., Arch. Biochem. Biophys., 259:52 (1987) and Edge et al., Anal. Biochem., 118:131 (1981)). Enzymatic cleavage of carbohydrate moieties on polypeptides can be achieved by the use of a variety of endo- and exo-glycosidases (see, for example, Thotakura et al., Meth. Enzymol. 138:350 (1987)).

Another type of covalent modification includes linking the compound to any of a variety of nonproteinaceous polymers, e.g., polyethylene glycol (PEG), polypropylene glycol (PPG), or polyoxyalkylenes (see, for example, U.S. Pat. Nos. 4,640,835; 4,496,689; 4,301,144; 4,670,417; 4,791,192 and 4,179,337).

NP-compounds of the invention can also be modified to form a chimeric molecule. In accordance with the invention, there are provided compounds that include a heterologous domain. A heterologous domain can be an addition or insertion. A heterologous domain can consist of any of a variety of different types of small or large functional moieties. Particular non-limiting examples of heterologous domains include, for example, tags, detectable labels and cytotoxic agents. Such moieties include peptides, nucleic acids, carbohydrate, lipid or small organic compounds, such as a drug (e.g., a cell proliferative agent), metals (gold, silver), etc.

Specific examples of tags and detectable labels include enzymes (horseradish peroxidase, urease, catalase, alkaline phosphatase, beta-galactosidase, chloramphenicol transferase); enzyme substrates; ligands (e.g., biotin); receptors (avidin); radionuclides (e.g., C¹⁴, S³⁵, P³², P³³, H³, I¹²⁵, I¹³¹, gallium-67 and 68, scantium-47, indium-111, radium-223); T7-, His-, myc-, HA- and FLAG-tags; electron-dense reagents; energy transfer molecules; paramagnetic labels; fluorophores (fluorescein, rhodamine, phycoerthrin); chromophores; chemi-luminescent (imidazole, luciferase); and bio-luminescent agents. Specific examples of cytotoxic agents include diptheria, toxin, cholera toxin and ricin.

In one embodiment, a heterologous domain comprises a peptide, heterologous polypeptide or an amino acid sequence fused to a compound. A chimeric molecule can include a fusion of the compound with a tag polypeptide which provides an epitope to which an anti-tag antibody can selectively bind. The epitope tag is generally placed at the end of the compound, e.g., at the amino- or carboxyl-terminus The epitope-tagged forms can be detected using an antibody against the tag polypeptide. Also, the epitope tag enables the compound to be readily purified by affinity purification using an anti-tag antibody or another type of affinity matrix that binds to the epitope tag. Various tag polypeptides and their respective antibodies are known to the skilled artisan. In an alternative embodiment, the chimeric molecule may comprise a fusion of the compound with an immunoglobulin or a particular region of an immunoglobulin. For a bivalent form of the chimeric molecule (also referred to as an “immunoadhesin”), such a fusion could be to the Fc region of an IgG molecule.

Additional examples of heterologous domains include, for example, anti-cell proliferative agents. Specific non-limiting examples of anti-cell proliferative agents are known to one of skill in the art.

Linker sequences may be inserted between the compound, and the heterologous domain so that the two entities maintain, at least in part, a distinct function or activity. Linker sequences may have one or more properties that include a flexible structure, an inability to form an ordered secondary structure or a hydrophobic or charged character which could promote or interact with either domain. Amino acids typically found in flexible protein regions include Gly, Asn and Ser. Other near neutral amino acids, such as Thr and Ala, may also be used in the linker sequence. The length of the linker sequence may vary (see, e.g., U.S. Pat. No. 6,087,329). Linkers further include chemical cross-linking and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST).

The term “isolated” used as a modifier of a compound means that the compound is manipulated or made by the hand of man or is separated from one or more other components in their naturally occurring in vivo environment. Generally, if such compounds exist naturally, the compounds are separated to be substantially free of one or more materials with which they normally associate with in nature, for example, one or more protein, nucleic acid, lipid, carbohydrate, cell membrane, etc. Thus, an isolated compound is substantially separated from other biological components in the cell of the organism in which the compound naturally occurs, or from the artificial medium in which it is produced (e.g., synthetically or through cell culture). For example, an isolated polypeptide is substantially separated from other polypeptides and nucleic acid and does not include a library of polypeptides or polynucleotides present among millions of polypeptide or nucleic acid sequences, such as a polypeptide, genomic or cDNA library, for example.

The term “isolated” does not exclude alternative physical forms of the compound, for example, an isolated peptide could include peptide multimers, disulfide linkages within or with other peptides, post-translational modifications (e.g., glycosylation, phosphorylation) and modified or derivatized forms. The term isolated is also intended not to exclude compounds of the invention that are present in a subject after administration. Such compounds in a subject can result from administration of the compound to the subject, or conversion of a prodrug form to a compound after the prodrug is administered to a subject. Thus, it is expressly intended that the compounds of the invention include prodrugs that convert to an invention compound subsequent to administration to a subject.

The term “purified” used as a modifier of a compound refers to a compound free of most or all of the materials with which it typically associates with in nature. Thus, a compound separated from cells is considered to be purified when separated from cellular components, while a chemically synthesized compound is considered to be substantially purified when separated from its chemical precursors. Purified therefore does not require absolute purity. Furthermore, a “purified” compound can be combined with one or more other molecules. Thus, the term “purified” does not exclude compound combinations.

“Purified” compounds include compounds produced by standard purification methods. The term also includes proteins and nucleic acids produced by recombinant expression in a host cell as well as chemical synthesis. “Purified” can also refer to a compound in which the level of contaminants is below a level that is acceptable to a regulatory agency for administration to a human or non-human animal, for example, the Food and Drug administration (FDA).

Substantial purity can be at least about 60% or more of the molecule by mass. Purity can also be about 70% or 80% or more, and can be greater, for example, 90% or more. Purity can be less, for example, in a pharmaceutical carrier the amount of a molecule by weight % can be less than 60% but the relative proportion of the compound compared to other components with which it is normally associated with will be greater. Purity can be determined by any appropriate method, including, for example, UV spectroscopy, chromatography (e.g., HPLC, gas phase), gel electrophoresis (e.g., silver or coomassie staining) and sequence analysis (peptide and nucleic acid).

The invention includes in vivo uses and methods. For example, a subject may be treated by administering, for example, a compound of the invention.

In accordance with the invention, there are provided methods of treating a retinal disorder or disease in a subject. Such methods can be practiced with any of the compounds of the invention set forth herein. In one embodiment, a method includes administering to a subject an amount of compound having a conserved motif, effective to treat the retinal disorder or disease in the subject.

As used herein, the terms “retinal disorder or disease” and grammatical variations thereof, refers to any undesirable, or abnormal condition that affects retina. A non-limiting representative cause of a retinal disorder or disease would be undesirable or abnormal blood vessel growth, proliferation, survival, invasion or extension that is greater than desired or is abnormal, such as compared to a reference amount of vessel that is of the same tissue but is not considered greater than desired or abnormal.

Retinal disorders and diseases cell (vessel) growth, proliferation and or invasion or extension into retina or other tissues. Such diseases and disorders include cell hyperplastic conditions characterized by undesirable, excessive or abnormal vessel size or numbers, or vessel invasion or extension, as well as vessel cell numbers, cell growth, cell proliferation, or cell survival. Specific examples of such disorders and diseases include extension or growth of choroidal vessels into the retina or surrounding tissues. Additional examples of such disorders and diseases include extension or growth of the existing retinal vasculature, or growth of choroidal vessels into the retina (also referred to as choroidal neovascularization; CNV).

In various embodiments, a method includes administering to a subject a compound of the invention in an amount effective to treat the retinal disorder or disease in the subject. In particular aspects, the disorder or disease is macular degeneration, proliferative diabetic retinopathy (PDR), retinal vein occlusion, retinopathy of prematuria, pseudoxanthoma elasticum, optic disc drusen, extreme myopia, or malignant myopic degeneration. In a further aspect, the macular degeneration is “wet” age-related macular degeneration (AMD).

Invention methods can be used to reduce or inhibit the retinal disorder or disease, reduce or inhibit vessel growth, survival or invasion into retina or nearby tissue, reduce or inhibit a symptom of a retinal disorder or disease. Thus, methods of the invention include, among other things, 1) reducing or inhibiting growth, proliferation, survival, mobility or invasiveness of blood vessels into retina or nearby tissues; 2) reversing or stabilizing growth, proliferation, survival, mobility or invasiveness of blood vessels into retina or nearby tissues; 3) reducing or inhibiting formation or establishment of new blood vessels in retina or nearby tissues; and 4) reducing or inhibiting one or more symptoms of a retinal disorder or disease.

As used herein, the terms “treat,” “treating,” “treatment” and grammatical variations thereof mean subjecting an individual patient to a protocol, regimen, process or remedy, in which it is desired to obtain a physiologic response or outcome in that patient. Since every treated patient may not respond to a particular treatment method, protocol, regimen, process or remedy, treatment does not require that the desired physiologic response or outcome be achieved in each and every patient or patient population. Accordingly, although a given treatment method, protocol, regimen, process or remedy, will provide a benefit to certain patients, certain patients, or a patient population may fail to respond or respond less than optimally or inadequately to treatment.

Administration methods of the invention may be practiced by any mode of administration or by any desired route including, for example, systemic, regional and local administration, such as at or near the site of a retinal disorder or disease. For example, one or more eyes of a subject who is suffering from or is in need of treatment for a retinal disorder or disease may be treated by local or regional treatment of the eye(s), such as by an injection (e.g., intravitreal injection), infusion, or topically.

Exemplary administration routes include injection (e.g., intravitreal injection), infusion, intravenous, intrarterial, intradermal, intramuscular, subcutaneous, intra-pleural, topical, transmucosal, intra-ocular, oral (alimentary) and mucosal. Particular routes of administration can be based in part upon the type retinal disorder or disease, as well as pharmacology, bioavailability, stability of the compound, etc.

Methods of the invention include, among other things, methods that provide a detectable or measurable improvement in a condition of a given subject, such as alleviating or ameliorating one or more causes, adverse (physical) symptoms or consequences associated with the retinal disorder or disease, i.e., a therapeutic benefit or a beneficial effect. A therapeutic benefit or beneficial effect is any objective or subjective, transient, temporary, or long-term improvement in the retinal disorder or disease, or a reduction in onset, severity, duration or frequency of an adverse symptom associated with or caused by retinal disorder or disease. A satisfactory clinical endpoint of a treatment method in accordance with the invention is achieved, for example, when there is an incremental or a partial reduction in severity, duration or frequency of one or more associated pathologies, adverse symptoms or complications, or inhibition or reversal of one or more of the physiological, biochemical or cellular manifestations or characteristics of the retinal disorder or disease. A therapeutic benefit or improvement therefore can be a cure, such as destruction of the retinal disorder or disease, or ablation of one or more, most or all pathologies, adverse symptoms or complications associated with or caused by the retinal disorder or disease. However, a therapeutic benefit or improvement need not be a complete cure or ablation of all pathologies, adverse symptoms or complications associated with or caused by a retinal disorder or disease. For example, slowing, or delaying progression or worsening of a retinal disorder or disease, a reduction or inhibition of progression or worsening of a retinal disorder or disease, a stabilization in a retinal disorder or disease, reduced or decreased numbers of blood vessels, or vessel invasiveness or extension (angiogenesis), such as in retina (e.g., extension or growth of choroidal vessels into retina) or other tissues, or reduced or inhibited vessel cell numbers, cell growth, cell proliferation, or cell survival, or reduction or inhibition in severity, frequency or duration of one or more symptoms associated with any of the foregoing that is elicited by NP compounds of the invention is included in the methods and uses of the invention.

Accordingly, in various embodiments, a method or use slows, or delays progression or worsening of a retinal disorder or disease, reduces or inhibits progression or worsening of a retinal disorder or disease, stabilizes a retinal disorder or disease, reduces or decreases numbers of blood vessels, or vessel invasiveness or extension (angiogenesis), such as in retina (e.g., extension or growth of choroidal vessels into retina) or other tissues, reduces or inhibits vessel cell numbers, cell growth, cell proliferation, or cell survival, or reduces or inhibits severity, frequency or duration of one or more symptoms associated with any of the foregoing.

An invention method may not take effect immediately. For example, treatment may be followed by no effect, or even an increase or worsening of the retinal disorder or disease, but over time in responsive subjects there will be a desired effect or activity on the retinal disorder or disease, as set forth herein or known to one of skill in the art.

Examination of a retina and/or vasculature (blood vessel density, length, amounts, etc.) can establish any effect of treatment or use of a compound of the invention. Other symptoms of retinal diseases and disorders can be ascertained in order to determine the effect, if any, of treatment. The occurrence of CNV and the effectiveness of its treatment can be monitored by fluorescent angiography, retinal tomography, fundus photography, and functional means such as visual field analysis or amsler grid testing.

Invention compounds and methods can be combined with any other treatment or therapy that provides a desired effect. In particular, treatments and therapies that have been characterized as having a beneficial activity or function in treatment of retinal diseases and disorders, including symptoms thereof, are applicable. Exemplary treatments and therapies include inhibitors and antagonists of vascular endothelial growth factor (VEGF), i.e., anti-VEGF regimens, agents and drugs, as well as anti-cell proliferative protocols, regimens, agents and drugs. Such treatments and therapies can be performed prior to, substantially contemporaneously with any other methods of the invention.

The invention therefore provides combination methods and uses, in which any of the NP compounds, are used in combination with, or administered in a combination (in series, sequentially, or simultaneously), with any therapeutic regimen, protocol or composition. In one embodiment, a method includes administering a compound having a conserved motif, and a VEGF antagonist or inhibitor to the subject. The vascular endothelial growth factor (VEGF) antagonist or inhibitor, or anti-cell proliferative treatment, agent or drug, can be administered prior to, substantially contemporaneously with, in a mixture with, or following administration of the compound.

As used herein, an “anti-cell proliferative,” treatment, therapy, activity or effect means any therapy, treatment regimen, agent, drug, protocol or process that is useful in treating pathologies, adverse symptoms or complications associated with or caused by abnormal or undesirable cell proliferation (hyperproliferation), a cellular hyperproliferative disorder, tumor, cancer or neoplasia, or metastasis. Examples of anti-cell proliferative treatments and therapies include chemotherapy, immunotherapy, radiotherapy (ionizing or chemical), local or regional thermal (hyperthermia) therapy and surgical resection.

Methods of the invention also include, among other things, methods that result in a reduced need or use of another treatment protocol or therapeutic regimen, process or remedy. For example, for a retinal disorder or disease, a method of the invention has a therapeutic benefit if in a given subject it results in a less frequent or reduced dose or elimination of another treatment, such as an anti-VEGF treatment or therapy.

The doses or “amount effective” or “amount sufficient” in a method of treatment or use is an amount desired to have an effect, such as a therapeutic benefit or improvement, which includes, for example, any objective or subjective alleviation or amelioration of one, several or all pathologies, adverse symptoms or complications associated with or caused by the retinal disorder or disease, to a measurable or detectable extent, although preventing, inhibiting or delaying a progression or worsening of the retinal disorder or disease, adverse symptom or complication, is a satisfactory outcome. Thus, in the case of a retinal disorder or disease, the amount will desirably be effective or sufficient to provide a therapeutic benefit to a given subject or to alleviate or ameliorate a pathology, adverse symptom or complication of the retinal disorder or disease in a given subject. The dose may be proportionally increased or reduced as indicated by the status of treatment or the particular retinal disorder or disease or any side effect(s) of the treatment or therapy.

Exemplary non-limiting amounts (doses) are in a range of about 0.1 mg/kg to about 100 mg/kg, and any numerical value or range or value within such ranges. Greater or lesser amounts (doses) can be administered, for example, 0.01-500 mg/kg, and any numerical value or range or value within such ranges. Additional exemplary non-limiting amounts (doses) range from about 0.5-50 mg/kg, 1.0-25 mg/kg, 1.0-10 mg/kg, and any numerical value or range or value within such ranges.

Methods of the invention may be practiced one or more times (e.g., 1-10, 1-5 or 1-3 times) per day, week, month, or year. The skilled artisan will know when it is appropriate to modify an administration regimen, such as to increase, or to reduce, delay or discontinue administration. An exemplary non-limiting dosage schedule is 1-7 times per week, for 1, 2, 3, 4, 5, 6, 7, 8, 9, 10, 15, 20 or more weeks, and any numerical value or range or value within such ranges.

Of course, as is typical for any treatment or therapy, different subjects will exhibit different responses to treatment and some may not respond or respond inadequately to a particular treatment method or use. Amounts effective or sufficient will therefore depend at least in part upon the disorder treated (e.g., the type, severity, stage, etc. of the retinal disorder or disease), the therapeutic effect desired, as well as the individual subject (e.g., the bioavailability within the subject, gender, age, etc.) and the subject's response to the treatment based upon genetic and epigenetic variation (e.g., pharmacogenomics).

The terms “subject” and “patient” are used interchangeably herein and refer to animals, typically mammals, such as humans, non-human primates (gorilla, chimpanzee, orangutan, macaque, gibbon), domestic animals (dog and cat), farm and ranch animals (horse, cow, goat, sheep, pig), laboratory and experimental animals (mouse, rat, rabbit, guinea pig). Subjects include disease model animals (e.g., such as mice, rats and non-human primates) for studying in vivo efficacy against a retinal disorder or disease. Human subjects include children, for example, newborns, infants, toddlers and teens, between the ages of 1 and 5, 5 and 10 and 10 and 18 years, young adults between the ages of 18 and 25, adults between 25 and 60 years, and the elderly, for example, between the ages of 60 and 65, 65 and 70 and 70 and 100 years.

Subjects include mammals (e.g., humans) in need of treatment, that is, they have a retinal disorder or disease. Subjects also include those at risk of having a retinal disorder or disease. Subjects further include a subject in need of a treatment or therapy for a retinal disorder or disease due to a clinical or lab diagnosis warranting such treatment, subjects undergoing a treatment or therapy for a retinal disorder or disease, and subjects having undergone a therapy or treatment for a retinal disorder or disease and are at risk of relapse or recurrence, including a subject that is in remission from a retinal disorder or disease but who may be at risk of relapse.

At risk subjects include those with a family history, genetic predisposition, or who have suffered a previous affliction with a retinal disorder or disease and are at risk of relapse or recurrence. At risk subjects include elderly subjects.

At risk subjects can therefore be treated in order to inhibit or reduce the likelihood of developing a retinal disorder or disease, or after having been cured or treated for a retinal disorder or disease, suffering a relapse or recurrence of the same or a different retinal disorder or disease. The result of such treatment can be to reduce the risk of developing retinal disorder or disease, or to prevent a retinal disorder or disease, or a pathology, adverse symptom or complication thereof in the treated at risk subject.

The invention further provides kits, including compounds of the invention, modified and variants forms, and pharmaceutical formulations, packaged into suitable packaging material, optionally in combination with instructions for using the kit components, e.g., instructions for performing a method of the invention. In various embodiments, a kit includes a compound of the invention and instructions are for treating a retinal disorder or disease.

The term “packaging material” refers to a physical structure housing the components of the kit. The packaging material can maintain the components sterilely, and can be made of material commonly used for such purposes (e.g., paper, corrugated fiber, glass, plastic, foil, ampules, etc.). The label or packaging insert can include appropriate written instructions, for example, practicing a method or use of the invention, e.g., treating a retinal disorder or disease. Thus, in additional embodiments, a kit includes a label or packaging insert including instructions for practicing a method or use of the invention in solution, in vitro, in vivo, or ex vivo.

Instructions can therefore include instructions for practicing any of the methods of the invention described herein. For example, invention NP compounds can be included in a container, pack, or dispenser together with instructions for administration to a subject to treat a retinal disorder or disease. Instructions may additionally include indications of a satisfactory clinical endpoint or any adverse symptoms or complications that may occur, storage information, expiration date, or any information required by regulatory agencies such as the Food and Drug Administration for use in a human subject.

The instructions may be on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (hard disk), optical CD such as CD- or DVD-ROM/RAM, magnetic tape, electrical storage media such as RAM and ROM and hybrids of these such as magnetic/optical storage media.

Invention kits can additionally include a buffering agent, a preservative, or a stabilizing agent. The kit can also include control components for assaying for activity, e.g., a control sample or a standard. Each component of the kit can be enclosed within an individual container or in a mixture and all of the various containers can be within single or multiple packages.

Compounds of the invention, and other compositions and methods of the invention can be included in or employ pharmaceutical formulations. Such pharmaceutical formulations are useful for treatment of, or administration or delivery to, a subject in vivo or ex vivo.

Pharmaceutical formulations include “pharmaceutically acceptable” and “physiologically acceptable” carriers, diluents or excipients. As used herein the terms “pharmaceutically acceptable” and “physiologically acceptable” include solvents (aqueous or non-aqueous), solutions, emulsions, dispersion media, coatings, isotonic and absorption promoting or delaying agents, compatible with pharmaceutical administration. Such formulations can be contained in a liquid; emulsion, suspension, syrup or elixir, or solid form; tablet (coated or uncoated), capsule (hard or soft), powder, granule, crystal, or microbead. Supplementary compounds (e.g., preservatives, antibacterial, antiviral and antifungal agents) can also be incorporated into the formulations.

Pharmaceutical formulations can be made to be compatible with a particular local, regional systemic, or tissue or organ system administration or delivery mode or route. Thus, pharmaceutical formulations include carriers, diluents, or excipients suitable for administration by particular routes. Specific non-limiting examples of routes of administration for compositions of the invention are by infusion or parenteral, e.g., intravenous, intrarterial, intradermal, intramuscular, subcutaneous, intra-pleural, topical, transmucosal, intra-cranial, intra-ocular, retinal, oral (alimentary), mucosal administration, and any other formulation suitable for the method or use.

Solutions or suspensions used for parenteral application can include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide.

Pharmaceutical formulations for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof. Fluidity can be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as mannitol, sorbitol, sodium chloride can be included in the composition. Including an agent which delays absorption, for example, aluminum monostearate or gelatin can prolong absorption of injectable compositions.

Sterile injectable formulations can be prepared by incorporating the NP compound in the required amount in an appropriate solvent with one or a combination of above ingredients. Generally, dispersions are prepared by incorporating the active compound into a sterile vehicle containing a basic dispersion medium and any other ingredient. In the case of sterile powders for the preparation of sterile injectable solutions, methods of preparation include, for example, vacuum drying and freeze-drying which yields a powder of the active ingredient plus any additional desired ingredient from a previously prepared solution thereof.

For transmucosal or transdermal administration, penetrants appropriate to the barrier to be permeated are used in the formulation. Such penetrants are known in the art, and include, for example, for transmucosal administration, detergents, bile salts, and fusidic acid derivatives. Transmucosal administration can be accomplished through the use of nasal sprays, inhalation devices (e.g., aspirators) or suppositories. For transdermal administration, NP compounds are formulated into ointments, salves, gels, creams or patches.

The pharmaceutical formulations can be prepared with carriers that protect against rapid elimination from the body, such as a controlled release formulation or a time delay material such as glyceryl monostearate or glyceryl stearate. The formulations can also be delivered using articles of manufacture such as implants and microencapsulated delivery systems to achieve local, regional or systemic delivery or controlled or sustained release.

Biodegradable, biocompatible polymers can be used, such as ethylene vinyl acetate, polyanhydrides, polyglycolic acid, collagen, polyorthoesters, and polylactic acid. Methods for preparation of such formulations are known to those skilled in the art. The materials can also be obtained commercially from Alza Corporation (Palo Alto, Calif.). Liposomal suspensions (including liposomes targeted to cells or tissues using antibodies or viral coat proteins) can also be used as pharmaceutically acceptable carriers. These can be prepared according to known methods, for example, as described in U.S. Pat. No. 4,522,811.

Additional pharmaceutical formulations appropriate for administration are known in the art (see, e.g., Gennaro (ed.), Remington: The Science and Practice of Pharmacy, 20^{th} ed., Lippincott, Williams & Wilkins (2000); Ansel et al., Pharmaceutical Dosage Forms and Drug Delivery Systems, 7^{th} ed., Lippincott Williams & Wilkins Publishers (1999); Kibbe (ed.), Handbook of Pharmaceutical Excipients American Pharmaceutical Association, 3^{rd} ed. (2000); and Remington's Pharmaceutical Principles of Solid Dosage Forms, Technonic Publishing Co., Inc., Lancaster, Pa., (1993)).

The compounds used in accordance with the invention, including pharmaceutical formulations can be packaged in dosage unit form for ease of administration and uniformity of dosage. “Dosage unit form” as used herein refers to physically discrete units suited as unitary dosages treatment; each unit contains a quantity of the compound in association with the carrier, excipient, diluent, or vehicle calculated to produce a desired treatment or therapeutic (e.g., beneficial) effect. The unit dosage forms will depend on a variety of factors including, but not necessarily limited to, the particular compound employed, the effect to be achieved, and the pharmacodynamics and pharmacogenomics of the subject to be treated.

In accordance with the invention, there are provided methods of identifying a compound having activity, as well as methods for measuring activity of a compound. In one embodiment, a method includes screening a compound for inhibition of cell proliferation. A compound that is determined to inhibit cell proliferation is a compound having anti-cell proliferative activity. In another embodiment, a method includes screening a compound for binding to NPRC or activation of NPRC. A compound that is determined to bind to NPRC or activate NPRC is a compound having anti-cell proliferative activity. In yet another embodiment, a method includes screening a compound for inhibition of vessel cell growth, proliferation, survival or invasion.

In order to calculate, evaluate or identify a given compound for activity or an amount of activity, a variety of assays can be used. For example, cell toxicity and viability (apoptosis, lysis, growth proliferation, etc.) can be measured on the basis of colorimetric, luminescent, radiometric, or fluorometric assays known in the art. Colorimetric techniques, for example, Trypan Blue exclusion can be used to determine cell viability. In brief, cells are stained with Trypan Blue and counted using a hemocytometer. Viable cells exclude the dye whereas dead and dying cells take up the blue dye and are easily distinguished under a light microscope. Neutral Red is adsorbed by viable cells and concentrates in cell lysosomes; viable cells can be determined with a light microscope by quantitating numbers of Neutral Red stained cells.

Fluorometric techniques for determining cell viability include, for example, propidium iodide, a fluorescent DNA intercalating agent. Propidium iodide is excluded from viable cells but stains the nucleus of dead cells. Flow cytometry of propidium iodide labeled cells can then be used to quantitate viable and dead cells. Release of lactate dehydrogenase (LDH) indicates structural damage and death of cells, and can be measured by a spectrophotometric enzyme assay. Bromodeoxyuridine (BrdU) is incorporated into newly synthesized DNA and can be detected with a fluorochrome-labeled antibody. The fluorescent dye Hoechst 33258 labels DNA and can be used to quantitate proliferation of cells (e.g., flow cytometry). Quantitative incorporation of the fluorescent dye carboxyfluorescein diacetate succinimidyl ester (CFSE or CFDA-SE) can provide cell division analysis (e.g., flow cytometry). This technique can be used either in vitro or in vivo. 7-aminoactinomycin D (7-AAD) is a fluorescent intercalator that undergoes a spectral shift upon association with DNA, and can provide cell division analysis (e.g., flow cytometry).

Radiometric techniques for ascertaining cell proliferation include, for example, [³H]-Thymidine, which is incorporated into newly synthesized DNA of living cells and frequently used to determine proliferation of cells. Chromium (⁵¹Cr)-release from dead cells can be quantitated by scintillation counting in order to quantitate cell viability.

Luminescent techniques for determining cell viability include, for example, the CellTiter-Glo luminescent cell viability assay (Promega Madison Wis.). This technique quantifies the amount of ATP present to determine the number of viable cells.

Commercially available kits for determining cell viability and cell proliferation, and therefore the extent of anti-cell proliferative activity of a compound of the invention, include, for example, Cell Proliferation Biotrak ELISA (Amersham Biosciences Piscataway, N.J.); the Guava ViaCount™ Assay, which provides rapid cell counts and viability determination based on differential uptake of fluorescent reagents (Guava Technologies, Hayward, Calif.); the CyQUANT® Cell Proliferation Assay Kit (Molecular Probes, Inc., Eugene, Oreg.); and the CytoLux Assay Kit (PerkinElmer Life Sciences Inc., Boston, Mass.). The DELFIA® Assay Kits (PerkinElmer Life Sciences Inc., Boston, Mass.) can determine cell proliferation and viability using a time-resolved fluorometric method. The Quantos™ Cell Proliferation Assay is a fluorescence-based assay that measures the fluorescence of a DNA-dye complex from lysed cells (Stratagene, La Jolla, Calif.). The CellTiter-Glo cell viability assay is a luminescent assay for measuring cell viability (Promega, Madison Wis.).

The term “contacting,” when used in reference to a composition such as a compound (e.g., a compound with a conserved motif having activity for treatment of a retinal disorder or disease), material, sample, or treatment, means a direct or indirect interaction between the compound and the other referenced entity. A particular example of direct interaction is binding to NPR. A particular example of an indirect interaction is where the compound acts upon an intermediary molecule, which in turn acts upon the referenced entity. Thus, for example, contacting a cell with a compound of the invention includes allowing the compound to bind to the cell, or allowing the compound to act upon an intermediary (e.g., receptor) that in turn acts upon the cell.

The terms “assaying” and “measuring” and grammatical variations thereof are used interchangeably herein and refer to either qualitative or quantitative determinations, or both qualitative and quantitative determinations. When the terms are used in reference to activity, any means of assessing the relative activity is contemplated, including the various methods set forth herein and known in the art.

In accordance with the invention, further provided are methods of producing compounds of the invention. In one embodiment, a method includes transforming a host cell with a nucleic acid encoding a compound and culturing the host cell under conditions that allow expression of the encoded compound, and optionally isolating or purifying the compound from the transformed cells.

Thus, there are also provided host cells that express compounds of the invention as set forth herein. In particular embodiments, a host cell transformed with a nucleic acid encoding a compound having a conserved motif which has anti-cell proliferative activity expresses the compound.

Unless otherwise defined, all technical and scientific terms used herein have the same meaning as commonly understood by one of ordinary skill in the art to which this invention relates. Although methods and materials similar or equivalent to those described herein can be used in the practice or testing of the invention, suitable methods and materials are described herein.

All publications, patents, Genbank accession numbers and other references cited herein are incorporated by reference in their entirety. In case of conflict, the present specification, including definitions, will control.

As used herein, singular forms “a”, “and,” and “the” include plural referents unless the context clearly indicates otherwise. Thus, for example, reference to a “compound” or a “peptide” or “mimetic” includes a plurality of compounds, peptides or mimetics, and reference to “a treatment or therapy” can include multiple simultaneous, consecutive or sequential treatments or therapies, and so forth.

As used herein, all numerical values or numerical ranges include whole integers within or encompassing such ranges and fractions of the values or the integers within or encompassing ranges unless the context clearly indicates otherwise. Thus, for example, reference to a range of 8 to 50, includes any numerical value or range within or encompassing such values, such as 8, 9, 10, 11, 12, 13, 14, 15, 16, 17, 18, 19, 20, 21, 22, 23, 24, 25, 26, 27, 28, 29, 30, etc.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed.

A number of embodiments of the invention have been described. Nevertheless, it will be understood that various modifications may be made without departing from the spirit and scope of the invention. Accordingly, the following examples are intended to illustrate but not limit the scope of invention described in the claims. For example, the following non-limiting examples include methods for ascertaining activity, as well as describe a limited number of exemplary peptides and methods of use. These exemplary compositions and methods of treatment do not limit the invention, but provide general guidance to prepare and use the compounds, compositions and methods of the invention.

The studies herein should therefore not be construed as specifically limiting the invention and such variations of the invention, now known or later developed, which would be within the purview of one skilled in the art are considered to fall within the scope of the invention as described herein and hereinafter claimed. For example, in each case of an exemplary peptide shown below, a compound of the invention may be substituted for the exemplary peptide.

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

### EXAMPLES

### Example 1

This example describes a general method of producing peptides, proteins, fragments and mimetics that include a conserved motif.

Desired peptide fragments may be chemically synthesized. An alternative approach involves generating peptide fragments by enzymatic digestion, e.g., by treating the protein with an enzyme that cleaves proteins at sites defined by particular amino acid residues. Another alternative approach involves genetic methodology. Such techniques include expression of all or a part of the gene encoding the compound into a host cell such as mammalian cell, HeLa or Cos cells or E. coli. Such host cells can express full length or a fragment, for example, an scFv (see, e.g., Whitlow et al., In: Methods: A Companion to Methods in Enzymology 2:97 (1991), Bird et al., Science 242:423 (1988); and U.S. Pat. No. 4,946,778). For example, digesting a DNA encoding a peptide compound with a suitable restriction enzyme, and isolating the desired fragment and insert it into a vector for cell expression of the encoded peptide compound. Yet another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR.

In preparing a peptide, variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the variant DNA.

### Example 2

This example describes data indicating that two exemplary peptides with a conserved motif have activity. This example also includes a description of an exemplary cell based assay for ascertaining anti-cell proliferative activity.

Human Pancreatic Adenocarcinoma Cells (HPAC) cells were obtained from ATCC and propagated in Dulbecco's modified Eagle's medium and Ham's F12 medium (DME-F12) (1:1) containing 2.5 mM glutamine, 2 mg/mL insulin, 5 ug/mL transferrin, 1 ng/mL EGF (epidermal growth factor), plus 5% FBS (fetal bovine serum). Cell cultures were incubated at 37° C., 5% CO₂ in a 100% humidity atmosphere. The cells were routinely brought to confluence in T-flasks, rinsed with phosphate buffered saline(PBS), and treated with trypsin for 5 min at room temp. The trypsinized cells were rinsed from the plate with 10 mL medium and counted. Viability at subculturing was generally ≧95%.

Approximately 10,000 cells in 100 uL of medium were placed in each well of a 96-well plate and incubated over night. Peptides having the following sequences were used: Arg-15-Cys (RSSCAGAALSPLGAC); Pro-15-Val (PNEEAGAALSPLPEV); Ala-8-Leu (AGALSPL); C-ANP 4-23 (RSSCFGGRIDRIGAC) and, VDL (EVVPPQVLSEPNEEAGAALSPLPEVPPWTGEVSPAQR). The peptides were dissolved in PBS and sterile filtered. The peptides were diluted into the cell culture medium to the following final concentrations: 1, 0.3, 0.1, 0.03, and 0.01 uM. Each peptide was tested in 5 replicate wells. The cultures were incubated for an additional 48 hours at 37° C.

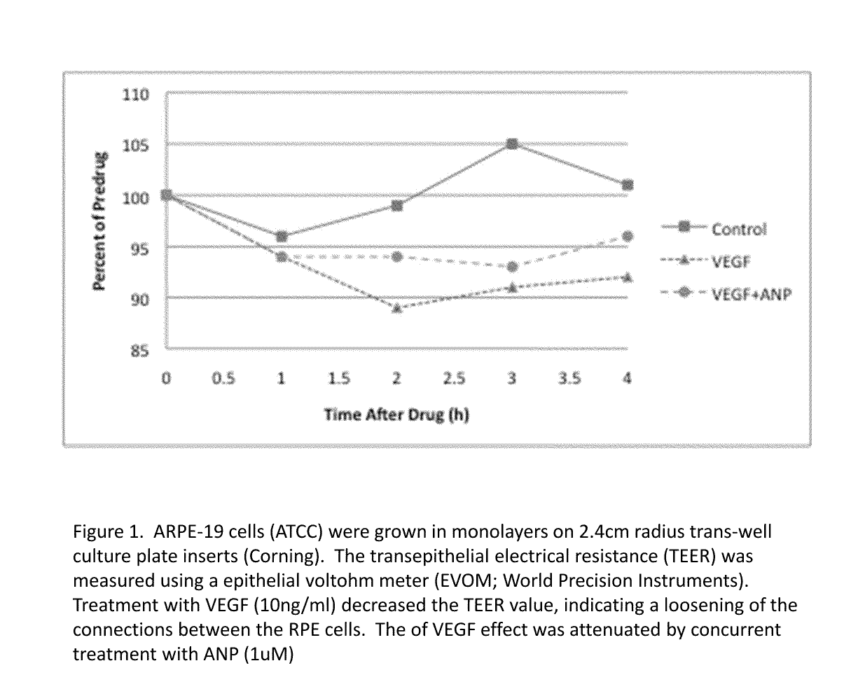

The viability of the cells was determined using a Calbiochem Rapid Cell Proliferation Kit (catalog number QIA127). Ten uL of Calbiochem color reagent was added to each well and incubated for an additional 1.5 hours at 37° C. Optical densities were read at 450 nm using a Molecular Devices ThermoMax microplate reader. The plate template is shown in Table I (unlabeled wells contained cells but no peptide and were used as blanks). Results are presented in Table I, and are graphically illustrated in FIGS. 1 and 2.

TABLE I
Sample Locations in 96-Well Plates

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

Plate 1

| | | | | | | | | | | | | |
|---|---|---|---|---|---|---|---|---|---|---|---|---|
| A | Arg-15- | 0.3 | 0.1 | 0.03 | 0.01 | | | 0.01 | 0.03 | 0.1 | 0.3 | 1 |
| | Cys 1 | | | | | | | | | | | |
| B | | 0.01 | 1 | 0.3 | 0.1 | 0.03 | 0.03 | 0.1 | 0.3 | 1 | 0.01 | |
| C | Pro-15- | 0.1 | 0.3 | 1 | 0.01 | | | 0.01 | 1 | 0.1 | 0.3 | 0.03 |
| | Val 0.03 | | | | | | | | | | | |
| D | | 0.01 | 0.03 | 0.1 | 0.3 | 1 | 1 | 0.3 | 0.1 | 0.03 | 0.01 | |
| E | | Ala-8- | 0.3 | 0.1 | 0.03 | 1 | 1 | 0.3 | 0.1 | 0.03 | 0.01 | |
| | | Leu 0.01 | | | | | | | | | | |
| F | 0.3 | 0.1 | 0.03 | 1 | 0.01 | | | 0.01 | 1 | 0.3 | 0.1 | 0.03 |
| G | | 0.01 | 1 | 0.03 | 0.1 | 0.3 | 0.03 | 0.1 | 0.3 | 1 | 0.01 | |
| H | | | | | | | | 0.01 | 0.03 | 0.1 | 0.3 | 1 |

Plate 2

| | | | | | | | | | | | | |
|---|---|---|---|---|---|---|---|---|---|---|---|---|
| A | ANP 1 | 0.3 | 0.1 | 0.03 | 0.01 | | | | | | | |
| B | | 0.01 | 1 | 0.3 | 0.1 | 0.03 | | | | | | |
| C | C-ANP 0.03 | 0.1 | 0.3 | 1 | 0.01 | | | | | | | |
| D | | 0.01 | 0.03 | 0.1 | 0.3 | 1 | 1 | 0.3 | 0.1 | 0.03 | 0.01 | |
| E | | 0.01 | 0.03 | 0.1 | 0.3 | 1 | 1 | 0.3 | 0.1 | 0.03 | 0.01 | |
| F | | | | | | | | 0.01 | 1 | 0.3 | 0.1 | 0.03 |
| G | | | | | | | 0.03 | 0.1 | 0.3 | 1 | 0.01 | |
| H | | | | | | | | 0.01 | 0.03 | 0.1 | 0.3 | 1 |

Numerical values indicate peptide micromolar concentrations

TABLE II
Optical Densities in 96_Well Plate

| Arg-15-Cys | 1.0 μM | 0.3 μM | 0.1 μM | 0.03 μM | 0.01 μM | | 0.0 μM | | 0.0 μM |
|---|---|---|---|---|---|---|---|---|---|
| | 1.155 | 0.988 | 1.016 | 1.093 | 0.974 | | 1.029 | | 0.866 |
| | 1.005 | 0.957 | 0.917 | 0.894 | 0.920 | | 0.858 | | 0.988 |
| | 0.787 | 0.668 | 0.801 | 0.789 | 0.863 | | 1.135 | | 1.033 |
| | 0.944 | 0.913 | 0.990 | 0.955 | 0.840 | | 1.279 | | 1.003 |
| | 0.919 | 0.959 | 1.025 | 1.178 | 1.075 | | 1.255 | | 0.881 |
| | | | | | | | 0.999 | | 1.013 |
| Average | 0.962 | 0.897 | 0.950 | 0.982 | 0.934 | | 1.050 | | 0.854 |
| Std dev | 0.134 | 0.131 | 0.093 | 0.155 | 0.094 | | | | |
| | | | | | | | 0.946 | | 0.826 |
| Pro-15-Val | 1.0 μM | 0.3 μM | 0.1 μM | 0.03 μM | 0.01 μM | | 0.922 | | 0.971 |
| | 0.856 | 0.875 | 0.680 | 0.892 | 0.907 | | 0.967 | | 1.055 |
| | 0.752 | 0.817 | 0.895 | 0.765 | 0.905 | | 0.804 | | 0.871 |
| | 0.802 | 0.855 | 0.893 | 0.693 | 0.633 | | 0.817 | | 0.893 |
| | 1.083 | 1.024 | 1.156 | 1.151 | 1.209 | | 0.966 | | 0.774 |
| | 1.248 | 1.010 | 0.964 | 0.970 | 0.928 | | 0.977 | | 0.933 |
| | | | | | | | 0.892 | | 1.014 |
| Average | 0.948 | 0.916 | 0.918 | 0.894 | 0.916 | | 0.962 | | 0.873 |
| Std dev | 0.210 | 0.094 | 0.171 | 0.179 | 0.204 | | | | 0.966 |
| | | | | | | | 0.974 | | 1.010 |
| Ala-8-Leu | 1.0 μM | 0.3 μM | 0.1 μM | 0.03 μM | 0.01 μM | | 0.847 | | 0.926 |
| | 0.732 | 0.784 | 0.832 | 0.892 | 0.744 | | 0.562 | | 0.897 |
| | 1.216 | 0.802 | 1.006 | 0.937 | 0.878 | | 0.941 | | 0.868 |
| | 0.954 | 0.998 | 1.031 | 0.989 | 1.000 | | 1.310 | | 0.934 |
| | 0.834 | 1.357 | 0.910 | 0.921 | 0.892 | | | | 0.988 |
| | 1.189 | 1.070 | 1.174 | 1.139 | 1.169 | Plate 1 Avg. | 0.976 | | 0.965 |
| | | | | | | Std dev | 0.171 | | 0.820 |
| Average | 0.985 | 1.002 | 0.991 | 0.976 | 0.937 | | | | 0.878 |
| Std dev | 0.214 | 0.234 | 0.130 | 0.098 | 0.159 | | | | 0.945 |
| | | | | | | | | | 1.005 |
| ANP | 1.0 μM | 0.3 μM | 0.1 μM | 0.03 μM | 0.01 μM | | | | 0.932 |
| | 1.018 | 1.001 | 0.884 | 0.948 | 0.981 | | | | 0.922 |
| | 0.819 | 0.889 | 0.889 | 0.882 | 0.815 | | | | 0.744 |
| | 0.954 | 0.837 | 0.907 | 0.911 | 0.822 | | | | 0.936 |
| | 0.829 | 0.934 | 0.862 | 0.951 | 0.965 | | | | 0.892 |
| | 0.951 | 1.209 | 0.650 | 0.982 | 0.955 | | | | 0.895 |
| | | | | | | | | | 0.906 |
| Average | 0.914 | 0.974 | 0.838 | 0.935 | 0.908 | | | | 0.914 |
| Std dev | 0.087 | 0.145 | 0.107 | 0.039 | 0.082 | | | | 0.849 |
| | | | | | | | | | 0.836 |
| C-ANP | 1.0 μM | 0.3 μM | 0.1 μM | 0.03 μM | 0.01 μM | | | | 0.877 |
| | 0.865 | 0.840 | 0.758 | 0.905 | 0.877 | | | | 0.828 |
| | 0.925 | 0.928 | 1.027 | 0.882 | 0.971 | | | | 1.016 |
| | 0.900 | 1.035 | 0.983 | 0.981 | 0.953 | | | | 0.909 |
| | 0.955 | 0.975 | 0.960 | 0.948 | 0.932 | | | | 0.911 |
| | 0.981 | 0.932 | 0.982 | 0.943 | 0.958 | | | | 1.015 |
| | | | | | | | | | 0.998 |
| Average | 0.925 | 0.942 | 0.942 | 0.932 | 0.938 | | | | 0.964 |
| Std dev | 0.045 | 0.071 | 0.106 | 0.039 | 0.037 | | | | |
| | | | | | | | | Plate 2 Avg. | 0.922 |
| | | | | | | | | Std dev | 0.071 |

### Example 3

This example describes activity of peptides having a conserved motif and conserved amino acids predicted to be functional in the motif. (Table III). This example also describes additional exemplary peptide sequences having a conserved motif predicted to have activity (Table IV).

TABLE III

| | Anti- | | | | | | | | | | | | | | | | | | |
|---|---|---|---|---|---|---|---|---|---|---|---|---|---|---|---|---|---|---|---|
| | cancer | | | | | | | | | | | | | | | | | | |
| Peptide | Activity | 1 | 2 | 3 | 4 | 5 | 6 | 7 | 8 | 9 | 10 | 11 | 12 | 13 | 14 | 15 | 16 | 17 | 18 |
| ANP | + | | | | | | | | S | L | R | R | S | S | C | F | G | G | R |
| CNP | + | | | | | | | | | | | | | | | F | G | L | K |
| KT-220 (VDL) | + | E | V | V | P | P | Q | V | L | S | E | P | N | E | E | A | G | A | A |
| LANP | + | | | | | | | | | | | | | | | F | K | N | L |
| KP | + | | | | | | | | | | | | | | | L | K | S | K |
| BNP | − | | | | | | | | | | | | | | | F | G | K | R |
| C-ANP 4-23 | + | | | | | | | | | | | R | S | S | C | F | G | G | R |
| Conserved Amino | | | | | | | | | | | | | | | | F, A, L | G, K | N, S, G, | Any |
| Acids | | | | | | | | | | | | | | | | | | L, A | |
| Peptides | | | | | | | | | | | | | | | | | | | |
| Pro-15-Val | + | | | | | | | | | | | P | N | E | E | A | G | A | A |
| Arg-15-Cys | + | | | | | | | | | | | R | S | S | C | A | G | A | A |
| 3 | N/D | | | | | | | | | | | R | S | S | C | A | G | K | A |
| Ala-8-Leu | | | | | | | | | | | | | | | | A | G | A | A |

| Peptide | 19 | 20 | 21 | 22 | 23 | 24 | 25 | 26 | 27 | 28 | 29 | 30 | 31 | 32 | 33 | 34 | 35 | 36 | 37 |
|---|---|---|---|---|---|---|---|---|---|---|---|---|---|---|---|---|---|---|---|
| ANP | M | D | R | I | G | A | Q | S | G | L | G | C | N | S | F | R | Y | | |
| CNP | L | D | R | I | | | | | | | | | | | | | | | |
| KT-220 (VDL) | L | S | P | L | P | E | V | P | P | W | T | G | E | V | S | P | A | Q | R |
| LANP | L | D | H | L | | | | | | | | | | | | | | | |
| KP | L | R | A | L | | | | | | | | | | | | | | | |
| BNP | M | D | R | I | | | | | | | | | | | | | | | |
| C-ANP 4-23 | I | D | R | I | G | A | C | | | | | | | | | | | | |
| Conserved Amino | L, M | S, D, R | Any | I, L | | | | | | | | | | | | | | | |
| Acids | | | | | | | | | | | | | | | | | | | |
| Peptides | | | | | | | | | | | | | | | | | | | |
| Pro-15-Val | L | S | P | L | P | E | V | | | | | | | | | | | | |
| Arg-15-Cys | L | S | P | L | G | A | C | | | | | | | | | | | | |
| 3 | L | S | P | L | G | A | C | | | | | | | | | | | | |
| Ala-8-Leu | L | S | P | L | | | | | | | | | | | | | | | |

Positions 15 to 21 in Table III correspond to Res1-Res8 of the conserved motif. Conserved amino acids at each of Residues 1-8 include, for example, the following: (15) F, A, L; (16) G,K; (17) N, S, G, L, A; (18) Any; (19) L, M; (20) S, D, R; (21) Any; (22) I, L. Conserved amino acids at each of Residues 1-8 include, for example, Res 1: a hydrophobic, non-polar, non-ionizable residue; Res2 a non-hydrophobic (amphiphillic or hydrophilic) residue; Res3, a non-ionizable residue or a residue with a small side chain (0-2 carbon backbone, with 3 and 4 carbons weaker); Res4, Any residue; Res5, a hydrophobic, non-polar, non-ionizable or aliphatic residue; Res6, a polar residue; Res7, Any residue; and Res8, a hydrophobic, non-polar, non-ionizable or aliphatic residue.

Notations “Res 1, Res 2” etc., herein refer by single letter abbreviation the amino acid in the exemplary peptide 8-mer. “Activity” can be determined by a cell proliferative assay, such as an assay described herein or known to one of skill in the art. Of course, peptides exemplified in Table IV can include the conserved motif peptide as part of a larger protein. The SEQ ID NO is the same as the peptide number.

TABLE IV

| SEQ | | | | | | | | | Pre- |
|---|---|---|---|---|---|---|---|---|---|
| ID | Res | Res | Res | Res | Res | Res | Res | Res | dicted |
| NO | 1 | 2 | 3 | 4 | 5 | 6 | 7 | 8 | Activity |
| 1 | F | G | N | A | L | S | P | I | + |
| 2 | F | G | S | R | L | D | S | I | + |
| 3 | F | G | G | N | L | R | T | I | + |
| 4 | F | G | L | D | L | D | W | I | + |
| 5 | F | G | A | C | L | R | Y | I | + |
| 6 | F | G | N | E | M | S | V | I | + |
| 7 | F | G | S | Q | M | D | A | I | + |
| 8 | F | G | G | G | M | R | R | I | + |
| 9 | F | G | L | H | M | S | N | I | + |
| 10 | F | G | A | I | M | D | D | I | + |
| 11 | F | K | N | L | L | R | C | I | + |
| 12 | F | K | S | K | L | S | E | I | + |
| 13 | F | K | G | M | L | D | Q | I | + |
| 14 | F | K | L | G | L | R | G | I | + |
| 15 | F | K | A | F | L | S | H | I | + |
| 16 | F | K | N | P | M | D | I | I | + |
| 17 | F | K | S | S | M | R | L | I | + |
| 18 | F | K | G | T | M | S | K | I | + |
| 19 | F | K | L | W | M | D | M | I | + |
| 20 | F | K | A | Y | M | R | G | I | + |
| 21 | A | G | N | V | L | S | F | I | + |
| 22 | A | G | S | A | L | D | P | I | + |
| 23 | A | G | G | R | L | R | S | I | + |
| 24 | A | G | L | N | L | S | T | I | + |
| 25 | A | G | A | D | L | D | W | I | + |
| 26 | A | G | N | C | M | R | Y | I | + |
| 27 | A | G | S | E | M | S | V | I | + |
| 28 | A | G | G | Q | M | D | A | I | + |
| 29 | A | G | L | G | M | R | R | I | + |
| 30 | A | G | A | H | M | S | N | I | + |
| 31 | A | K | N | I | L | D | D | I | + |
| 32 | A | K | S | L | L | R | C | I | + |
| 33 | A | K | G | K | L | S | E | I | + |
| 34 | A | K | L | M | L | D | Q | I | + |
| 35 | A | K | A | G | L | R | G | I | + |
| 36 | A | K | N | F | M | S | H | I | + |
| 37 | A | K | S | P | M | D | I | I | + |
| 38 | A | K | G | S | M | R | L | I | + |
| 39 | A | K | L | T | M | S | K | I | + |
| 40 | A | K | A | W | M | D | M | I | + |
| 41 | L | G | N | Y | L | R | G | I | + |
| 42 | L | G | S | V | L | S | F | I | + |
| 43 | L | G | G | A | L | D | P | I | + |
| 44 | L | G | L | R | L | R | S | I | + |
| 45 | L | G | A | N | L | S | T | I | + |
| 46 | L | G | N | D | M | D | W | I | + |
| 47 | L | G | S | C | M | R | Y | I | + |
| 48 | L | G | G | E | M | S | V | I | + |
| 49 | L | G | L | Q | M | D | A | I | + |
| 50 | L | G | A | G | M | R | R | I | + |
| 51 | L | K | N | H | L | S | N | I | + |
| 52 | L | K | S | I | L | D | D | I | + |
| 53 | L | K | G | L | L | R | C | I | + |
| 54 | L | K | L | K | L | S | E | I | + |
| 55 | L | K | A | M | L | D | Q | I | + |
| 56 | L | K | N | G | M | R | G | I | + |
| 57 | L | K | S | F | M | S | H | I | + |
| 58 | L | K | G | P | M | D | I | I | + |
| 59 | L | K | L | S | M | R | L | L | + |
| 60 | L | K | A | T | M | S | K | L | + |
| 61 | F | G | N | W | L | S | M | L | + |
| 62 | F | G | S | Y | L | D | G | L | + |
| 63 | F | G | G | V | L | R | F | L | + |
| 64 | F | G | L | A | L | D | P | L | + |
| 65 | F | G | A | R | L | R | S | L | + |
| 66 | F | G | N | N | M | S | T | L | + |
| 67 | F | G | S | D | M | D | W | L | + |
| 68 | F | G | G | R | M | D | R | I | + |
| 69 | F | G | L | E | M | S | V | L | + |
| 70 | F | G | A | Q | M | D | A | L | + |
| 71 | F | K | N | G | L | R | R | L | + |
| 72 | F | K | S | H | L | S | N | L | + |
| 73 | F | K | G | I | L | D | D | L | + |
| 74 | F | K | L | L | L | R | C | L | + |
| 75 | F | K | A | K | L | S | E | L | + |
| 76 | F | K | N | M | M | D | Q | L | + |
| 77 | F | K | S | G | M | R | G | L | + |
| 78 | F | K | G | F | M | S | H | L | + |
| 79 | F | K | L | P | M | D | I | L | + |
| 80 | F | K | A | S | M | R | L | L | + |
| 81 | A | G | N | T | L | S | K | L | + |
| 82 | A | G | S | W | L | D | M | L | + |
| 83 | A | G | G | Y | L | R | G | L | + |
| 84 | A | G | L | V | L | S | F | L | + |
| 85 | A | G | A | A | L | S | P | L | + |
| 86 | A | G | N | R | M | R | S | L | + |
| 87 | A | G | S | N | M | S | T | L | + |
| 88 | A | G | G | D | M | D | W | L | + |
| 89 | A | G | L | C | M | R | Y | L | + |
| 90 | A | G | A | E | M | S | V | L | + |
| 91 | A | K | N | Q | L | D | A | L | + |
| 92 | A | K | S | G | L | R | R | L | + |
| 92 | A | K | G | H | L | S | N | L | + |
| 94 | A | K | L | I | L | D | D | L | + |
| 95 | A | K | A | L | L | R | C | L | + |
| 96 | A | K | N | K | M | S | E | L | + |
| 97 | A | K | S | M | M | D | Q | L | + |
| 98 | A | K | G | G | M | R | G | L | + |
| 99 | A | K | L | F | M | S | H | L | + |
| 100 | A | K | A | P | M | D | I | L | + |
| 101 | L | G | N | S | L | R | L | L | + |
| 102 | L | G | S | T | L | S | K | L | + |
| 103 | L | G | G | W | L | D | M | L | + |
| 104 | L | G | L | Y | L | R | G | L | + |
| 105 | L | G | A | V | L | S | F | L | + |
| 106 | L | G | N | A | M | D | P | L | + |
| 107 | L | G | S | R | M | R | S | L | + |
| 108 | L | G | G | N | M | S | T | L | + |
| 109 | L | G | L | D | M | D | W | L | + |
| 110 | L | G | A | C | M | R | Y | L | + |
| 111 | L | K | N | E | L | S | V | L | + |
| 112 | L | K | S | Q | L | D | A | L | + |
| 113 | L | K | G | G | L | R | R | L | + |
| 114 | L | K | L | H | L | S | N | L | + |
| 115 | L | K | A | I | L | D | D | L | + |
| 116 | L | K | N | L | M | R | C | L | + |
| 117 | L | K | S | K | M | S | E | L | + |
| 118 | L | K | G | M | M | D | Q | L | + |
| 119 | L | K | L | G | M | R | G | L | + |
| 120 | L | K | A | F | M | S | H | L | + |

Each of these exemplary peptides can be analyzed in vitro, ex vivo, and in vivo for anti-cell proliferative activity using an assay, such as a cell based in vitro assay described in Examples 1 or 4.

### Example 4

This example includes a description of another exemplary cell based assay to determine anti-cell proliferative activity.

Human ovarian carcinoma cells are cultured, and the peptides tested according to methods previously described [Vesely, et al, Cancer Ther. 5:97-104 (2007)]. To investigate whether the peptides inhibit DNA synthesis, and indication of cell proliferation, bromodeoxyuridine (BrdU) incorporation into the human ovarian carcinoma cells was utilized as previously described. [Vesely, D. L. Eur J Clin Invest. 38(8) 562:(2008) and references therein). BrdU was from BD Bioscience, San Jose, Calif. After 24 hours in culture with 1 μM of vessel dilator VDL, or with no peptide hormone (i.e., control), BrdU in a final concentration of 10 μM in the cell culture medium was added for 45 minutes, which is the time in which the cells are in the logarithmic phase of cell proliferation. In addition, if the addition of the peptide of the invention significantly decreased the number of ovarian cancer cells in 24 hours, compared to the control.

### Example 5

This example includes a description of testing of ANPs for NPR affinity as a proxy for activity.

Although not wishing to be bound by theory, the relative potencies of NP's are predicted to be related to binding affinities at NPRs [Vesely, D. L., Eur J Clin Invest. 38(8) 562:(2008) and references therein]. For example, the greater potency observed for ANP than CNP correlates with ANP's higher binding affinities at NPRA and NPRC, whereas the greater potency of CNP than BNP agrees with CNP having a higher binding affinity than BNP specifically at NPRC [Vesely, D. L., Eur J Clin Invest. 38(8) 562:(2008) and references therein].

The receptor selectivity of the natriuretic peptide family (ANP, BNP, CNP) using the homologous assay system described by Suga, et al., Endocrinol. 130:229-239 (1992). The rank order of binding affinity for the C-receptor follow the trends of the cell based assay of Example 4. Thus, binding affinities at NPRs, such as NPRC may be a good proxy for activity.

### Example 6

This example includes a description of peptidomimetics having the conserved motif.

Literature compounds, which appear to be peptidomimetics of the motif are assayed for cell-proliferative inhibiting activity (e.g., Examples 1 and 4) or are assayed for NPR binding (e.g., Example 5), and demonstrate binding. Predicted peptidomimetics include:

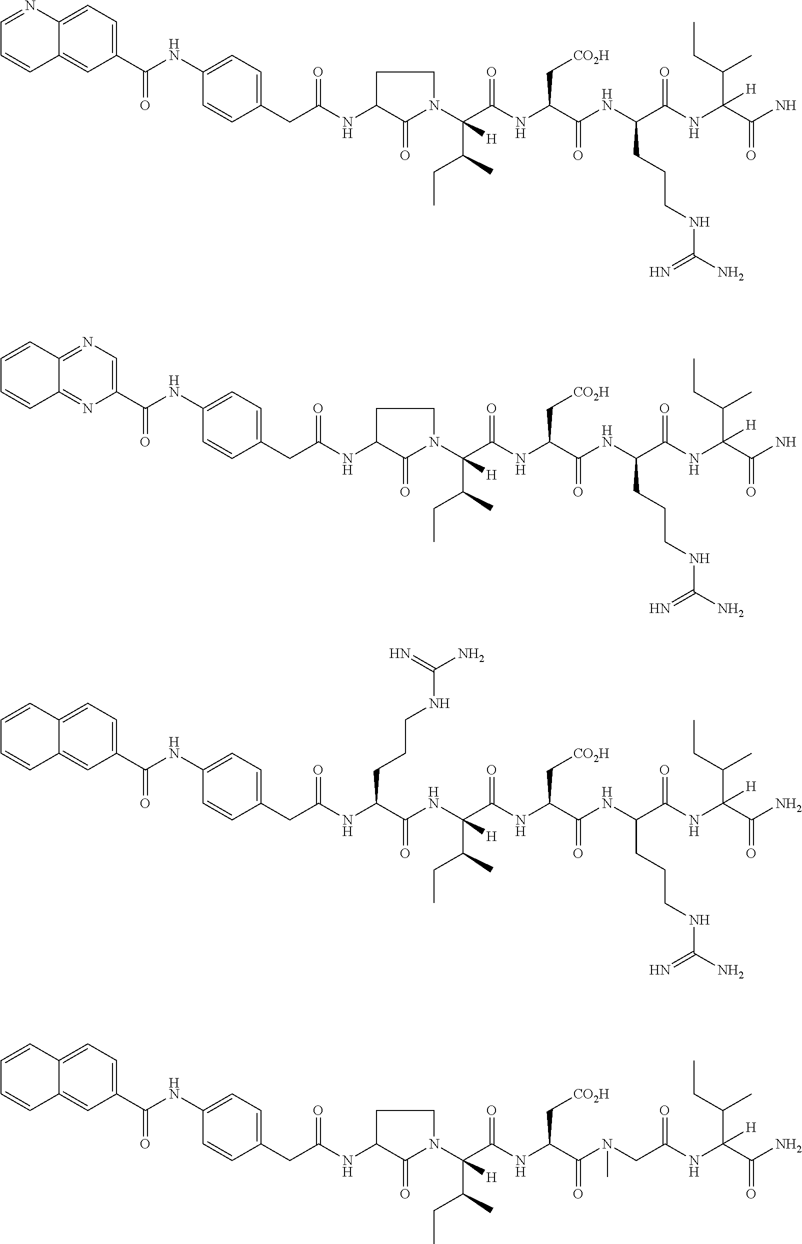

### Example 7

This example includes a description of various acceptable routes of systemic, regional or local, administration.

For administration by injection, an injectable solution can be prepared by conventional methods using 10.0 ml of physiological saline solution and 7.0 mg of a peptide adjusted to pH=7.4. One injection, one time daily for 4 days, can be administered to patients weighing approximately 70 kilogram.

For administration by infusion, an intravenous infusion composition can be prepared by conventional methods using 1000.0 ml of physiological saline solution and 400.0 mg of the peptide of Example 1 adjusted to pH=7.4. A one hour infusion, one time daily for 4 weeks, can be administered to a patient weighing approximately 58 kilograms.

For continuous administration, such as administration by Subcutaneous Injection via a pump, an injectable solution can be prepared by conventional methods using 1000.0 ml of physiological saline solution and 400.0 mg of a peptide adjusted to pH=7.4. The patient has a pump implanted to dispense the composition over time, as described in Vesely, D. L., In vivo 21:445-452 (2007). The implanted continuous infusion pump dispenses the composition at intervals, thrice daily for up to 8 weeks, to a patient weighing approximately 47 kilograms.

### Example 8

This example includes a description of studies to show the activity of NP compounds in treatment of retinal disorders and disease using an in vitro model system.

Human RPE cells (ARPE-19) were obtained from ATCC and cultured in DMEM-F12 with 10% FBS according to the supplier's instructions. The cells were plated into 2.4 cm radius transwell inserts in 6 well microtiter plates (Corning) and allowed to grow to confluence. After 2-3 weeks, voltage (transepithelial electrical resistance, TEER) was measured across the cell layer using a using an epithelial voltohmmeter (EVOM) with a STX2 electrode (World Precision Instruments) as previously described [Ablonczy, Z., Crosson, C. E. Exp Eye Res 85:762 (2007)]. Treatment of the cells with VEGF (10 ng/ml) lowered resistance, mimicking the disruption of the RPE layer that occurs in AMD (FIG. 1). Concurrent treatment with ANP (luM) attenuated the effect of VEGF. Other NP compounds can be evaluated in place of ANP to establish activity in AMD.

### Example 9

This example includes a description of studies to show activity of NP compounds in treatment of retinal disorders and disease using a rodent model system Noda, K., Nakazawa, T. et al, FASEB J; 22:2928 (2000).

CNV is induced in animals, generally mice or rats, under anesthesia using laser light with a slit-lamp delivery system, and a cover glass as a contact lens. Several laser burns are placed on each retina. The development of a bubble at the spot of the laser injury confirms the rupture of the Bruch's membrane. Seven days after laser injury, the size of the CNV lesions can be measured in situ, using slit lamp examination or fundus photography, or in choroidal flat mounts. Agents which prevent or reverse the formation of CNV will reduce the size of the lesion.

### Example 10

This example includes a description of studies to show the activity of NP compounds in the treatment of retinal disorders and disease using genetic rodent models.

A certain percentage of Senescence Accelerated Mice (SAM) spontaneously develop CNV as well as other manifestations of AMD after about 11 months of life (Majji, A. B., Cao, J. et al, Invest Ophthal Vis Sci; 41:3936 (2000). NP compounds can be administered to these animals to prevent or reverse the CNV. Alternatively, the RPE cells themselves can be genetically engineered to overexpress VEGF. When reintroduced subretinally into the rat eye, they produce CNV (Spilsbury, K. Garrett, K. L. et al, Am J Pathol; 157:135 (2000). Again, the NP compounds can be administered to these animals, and their effects on the development and persistence of the CNV determined

What is claimed is:

1. A method of treating a retinal disorder or disease in a subject in need of treatment, comprising administering to the subject a compound, wherein the compound comprises a conserved motif: (Res1)-(Res 2)-(Res 3)-(Res 4)-(Res 5)-(Res 6)-(Res 7)-(Res 8), wherein:
Res 1—is an amino acid or mimetic thereof, wherein the backbone is selected from an ether, ester, ketone, alkyl, alkylene or amide linker of 5 or less atoms, having a side chain selected from hydrophobic, non-polar, and non-ionizable side chain, selected from benzyl, alkyl, alkylene, alkenyl, alkylaryl of less than 12 carbons;
Res 2—is an amino acid or mimetic thereof, wherein the backbone is selected from an ether, ester, ketone, alkyl, alkylene or amide linker of 5 or less atoms, having a side chain which is an amphiphilic side chain, hydrophilic side chain, a zwitterion, Glycine (G), Lysine (K), Arginine (R), glutamine (Q), and Asparagine (N);
Res 3—is an amino acid or mimetic thereof, wherein the backbone is selected from an ether, ester, ketone, alkyl, alkylene or amide linker of 5 or less atoms, having a side chain less than 5 members long, except that any ring may be 5 or 6 members in size, and Glycine (G), Histidine (H) Asparagine (N), Serine (S), Leucine (L), Alanine (A)
Res 4—is a linker of 5 or less atoms or an amino acid, an ether, ester, ketone, alkyl, alkylene, amide linker, with a side chain comprising from 1 to about 12 carbons and with constiuents thereon selected from amino, hydroxyl, amido, carboxy, aryl, heteroaryl and any of the naturally occurring 20 amino acids;
Res 5—is an amino acid or mimetic thereof, wherein the backbone is selected from an ether, ester, ketone, alkyl, alkylene or amide linker of 5 or less atoms, having a side chain selected from a hydrophobic, non-polar, non-ionizable, aliphatic, side chain, and Leucine (L), Isoleucine (I), Valine (V), Alanine (A), or Methionine (M).
Res 6—is an amino acid or mimetic thereof, wherein the backbone is selected from an ether, ester, ketone, alkyl, alkylene or amide linker 5 or less atoms, having a polar side chain, or amino acids Serine (S), Aspartic Acid (D), Arginine (R), and Glutamic Acid (E)
Res 7—is an amino acid or mimetic thereof, wherein the backbone is selected from an ether, ester, ketone, alkyl, alkylene or amide linker 5 or less atoms, with a side chain comprising from 1 to about 12 carbons and with constiuents thereon selected from amino, hydroxyl, amido, carboxy, aryl, heteroaryl and any of the naturally occurring 20 amino acids;
Res 8—is an amino acid or mimetic thereof, wherein the backbone is selected from an ether, ester, ketone, alkyl, alkylene or amide linker of 5 or less atoms, having a hydrophobic, non-polar, and non-ionizable side chain, and amino acids Alanine (A), Leucine (L), Isoleucine (I), Valine (V), having a side chain selected from C1 to C 12 alkyl, alkylene, and alkenyl; and
wherein each of Res 1, Res 2, Res 3, Res 4, Res 5, Res 6, Res 7, and Res 8 are covalently or non-covalently bound to the respective adjacent residue, and wherein the compound has less than about 30 residues.

2. The method of claim 1, wherein:
Res 1—is selected from Phenylalanine (F), Alanine (A), Leucine (L), Isoleucine (I), and Valine (V);
Res 2—is selected from Glycine (G), Lysine (K), Arginine (R), glutamine (Q), and Asparagine (N);
Res 3 is selected from Glycine (G), Histidine (H) Asparagine (N), Serine (S), Leucine (L), and Alanine (A);
Res 4—is selected from any amino acid;
Res 5—is selected from Leucine (L), Isoleucine (I), Valine (V), Alanine (A), and Methionine (M);
Res 6—is selected from Serine (S), Aspartic Acid (D), Arginine (R), and Glutamic Acid (E);
Res 7—is selected from any amino acid; and
Res 8—is selected from Alanine (A), Leucine (L), Isoleucine (I), and Valine (V).

3. The method of claim 1, wherein:
Res 1—is selected from Phenylalanine (F), Alanine (A), and Leucine (L);
Res 2—is selected from Glycine (G) and Lysine (K);
Res 3 is selected from Glycine (G), Asparagine (N), Serine (S), Leucine (L) and Alanine (A);
Res 4—is selected from a natural amino acid;
Res 5—is selected from Leucine (L) and Methionine (M);
Res 6—is selected from Serine (S), Aspartic Acid (D), and Arginine (R);
Res 7—is selected from a natural amino acid; and
Res 8—is selected from Leucine (L) and Isoleucine (I).

4. The method of claim 3, wherein: Res 4—is selected from Arginine (R), Lysine (K), Alanine (A), and Leucine (L); and/or Res 7—is selected from Arginine (R), Proline (P), Histidine (H), and Alanine (A).

5. The method of claim 1, wherein the compound comprises: FGGRMDRI; FGLKLDRI, AGAALSPL, FKNLLDHL, LKSKLRAL, FGKRMDRI, FGGRIDRI, AGAALSPL, OR AGKALSPL.

6. The method of claim 1, wherein the compound comprises 1, 2, 3, 4, 5, 6, 7, 8, 9, 10, 11, 12, 13, 14, 15, 16, 17, 18, 19, 20, 21, 22, 23, 24, 25, 26, 27, 28, 29 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 856, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141 or 142 amino acid residues that includes the (Res1)-(Res 2)-(Res 3)-(Res 4)-(Res 5)-(Res 6)-(Res 7)-(Res 8) motif, or is covalently or non-covalently bonded to any of the Res 1, Res 2, Res 3, Res 4, Res 5, Res 6, Res 7, or Res 8 positions.

7. The method of claim 1, wherein the compound has less than about 150 residues.

8. The method of claim 1, wherein the compound has less than about 140 residues.

9. The method of claim 1, wherein the compound has between about 10 and 150 residues.

10. The method of claim 1, wherein the compound has between about 10 and 125 residues.

11. The method of claim 1, wherein the compound has between about 10 and 100 residues.

12. The method of claim 1, wherein any of Res 1, Res 2, Res 3, Res 4, Res 5, Res 6, Res 7, or Res 8 positions of the compound is an L-amino acid, a D-amino acid, a non-naturally occurring amino acid, or an amino acid derivative or analog.

13. The method of claim 1, wherein the retinal disorder or disease comprises macular degeneration, proliferative diabetic retinopathy (PDR), retinal vein occlusion, retinopathy of prematuria, pseudoxanthoma elasticum, optic disc drusen, extreme myopia, or malignant myopic degeneration.

14. The method of claim 13, wherein the macular degeneration comprises “wet” age-related macular degeneration (AMD).

15. The method of claim 1, wherein the retinal disorder or disease is caused by or associated with extension or growth of choroidal vessels into the retina.

16. The method of claim 1, wherein the retinal disorder or disease is caused by or associated with extension of the existing retinal vasculature, or growth of choroidal vessels into the retina (choroidal neovascularization; CNV).

17. The method of claim 1, wherein the retinal disorder or disease is progressively worsening.

18. The method of claim 1, wherein the retinal disorder or disease is in remission.

19. The method of claim 1, wherein the compound is administered to the subject locally, regionally, or systemically.

20. The method of claim 1, wherein the compound is administered to the subject's eye or eyes.

21. The method of claim 1, wherein the compound is administered by injection, infusion, orally or topically.

22. The method of claim 1, wherein the compound is administered to the subject via intravitreal injection.

23. The method of claim 1, wherein the treatment reduces or inhibits severity or duration of one or more symptoms of the retinal disorder or disease, or reduces or inhibits progression or worsening of the retinal disorder or disease.

24. The method of claim 1, wherein the treatment reduces or inhibits extension or growth of choroidal vessels into the retina of the subject.

25. The method of claim 1, wherein the subject is a candidate for, is undergoing, or has undergone a treatment or therapy for a retinal disorder.

26. The method of claim 1, further comprising administering to the subject a vascular endothelial growth factor (VEGF) antagonist or inhibitor to the subject.

27. The method of claim 26, wherein the compound is administered prior to, substantially contemporaneously with, in a mixture with, or following administration of the vascular endothelial growth factor (VEGF) antagonist or inhibitor.

28. The method of claim 1, wherein the subject is a mammal.

29. The method of claim 28, wherein the subject is a human.


SEQUENCE LISTING (132 sequences)

SEQ ID NO: 1 - protein, 8 aa, Artificial Sequence
  Description of Artificial Sequence Synthetic Peptide
FGNALSPI

SEQ ID NO: 2 - protein, 8 aa, Artificial Sequence
  Description of Artificial Sequence Synthetic Peptide
FGSRLDSI

SEQ ID NO: 3 - protein, 8 aa, Artificial Sequence
  Description of Artificial Sequence Synthetic Peptide
FGGNLRTI

SEQ ID NO: 4 - protein, 8 aa, Artificial Sequence
  Description of Artificial Sequence Synthetic Peptide
FGLDLDWI

SEQ ID NO: 5 - protein, 8 aa, Artificial Sequence
  Description of Artificial Sequence Synthetic Peptide
FGACLRYI

SEQ ID NO: 6 - protein, 8 aa, Artificial Sequence
  Description of Artificial Sequence Synthetic Peptide
FGNEMSVI

SEQ ID NO: 7 - protein, 8 aa, Artificial Sequence
  Description of Artificial Sequence Synthetic Peptide
FGSQMDAI

SEQ ID NO: 8 - protein, 8 aa, Artificial Sequence
  Description of Artificial Sequence Synthetic Peptide
FGGGMRRI

SEQ ID NO: 9 - protein, 8 aa, Artificial Sequence
  Description of Artificial Sequence Synthetic Peptide
FGLHMSNI

SEQ ID NO: 10 - protein, 8 aa, Artificial Sequence
  Description of Artificial Sequence Synthetic Peptide
FGAIMDDI

SEQ ID NO: 11 - protein, 8 aa, Artificial Sequence
  Description of Artificial Sequence Synthetic Peptide
FKNLLRCI

SEQ ID NO: 12 - protein, 8 aa, Artificial Sequence
  Description of Artificial Sequence Synthetic Peptide
FKSKLSEI

SEQ ID NO: 13 - protein, 8 aa, Artificial Sequence
  Description of Artificial Sequence Synthetic Peptide
FKGMLDQI

SEQ ID NO: 14 - protein, 8 aa, Artificial Sequence
  Description of Artificial Sequence Synthetic Peptide
FKLGLRGI

SEQ ID NO: 15 - protein, 8 aa, Artificial Sequence
  Description of Artificial Sequence Synthetic Peptide
FKAFLSHI

SEQ ID NO: 16 - protein, 8 aa, Artificial Sequence
  Description of Artificial Sequence Synthetic Peptide
FKNPMDII

SEQ ID NO: 17 - protein, 8 aa, Artificial Sequence
  Description of Artificial Sequence Synthetic Peptide
FKSSMRLI

SEQ ID NO: 18 - protein, 8 aa, Artificial Sequence
  Description of Artificial Sequence Synthetic Peptide
FKGTMSKI

SEQ ID NO: 19 - protein, 8 aa, Artificial Sequence
  Description of Artificial Sequence Synthetic Peptide
FKLWMDMI

SEQ ID NO: 20 - protein, 8 aa, Artificial Sequence
  Description of Artificial Sequence Synthetic Peptide
FKAYMRGI

SEQ ID NO: 21 - protein, 8 aa, Artificial Sequence
  Description of Artificial Sequence Synthetic Peptide
AGNVLSFI

SEQ ID NO: 22 - protein, 8 aa, Artificial Sequence
  Description of Artificial Sequence Synthetic Peptide
AGSALDPI

SEQ ID NO: 23 - protein, 8 aa, Artificial Sequence
  Description of Artificial Sequence Synthetic Peptide
AGGRLRSI

SEQ ID NO: 24 - protein, 8 aa, Artificial Sequence
  Description of Artificial Sequence Synthetic Peptide
AGLNLSTI

SEQ ID NO: 25 - protein, 8 aa, Artificial Sequence
  Description of Artificial Sequence Synthetic Peptide
AGADLDWI

SEQ ID NO: 26 - protein, 8 aa, Artificial Sequence
  Description of Artificial Sequence Synthetic Peptide
AGNCMRYI

SEQ ID NO: 27 - protein, 8 aa, Artificial Sequence
  Description of Artificial Sequence Synthetic Peptide
AGSEMSVI

SEQ ID NO: 28 - protein, 8 aa, Artificial Sequence
  Description of Artificial Sequence Synthetic Peptide
AGGQMDAI

SEQ ID NO: 29 - protein, 8 aa, Artificial Sequence
  Description of Artificial Sequence Synthetic Peptide
AGLGMRRI

SEQ ID NO: 30 - protein, 8 aa, Artificial Sequence
  Description of Artificial Sequence Synthetic Peptide
AGAHMSNI

SEQ ID NO: 31 - protein, 8 aa, Artificial Sequence
  Description of Artificial Sequence Synthetic Peptide
AKNILDDI

SEQ ID NO: 32 - protein, 8 aa, Artificial Sequence
  Description of Artificial Sequence Synthetic Peptide
AKSLLRCI

SEQ ID NO: 33 - protein, 8 aa, Artificial Sequence
  Description of Artificial Sequence Synthetic Peptide
AKGKLSEI

SEQ ID NO: 34 - protein, 8 aa, Artificial Sequence
  Description of Artificial Sequence Synthetic Peptide
AKLMLDQI

SEQ ID NO: 35 - protein, 8 aa, Artificial Sequence
  Description of Artificial Sequence Synthetic Peptide
AKAGLRGI

SEQ ID NO: 36 - protein, 8 aa, Artificial Sequence
  Description of Artificial Sequence Synthetic Peptide
AKNFMSHI

SEQ ID NO: 37 - protein, 8 aa, Artificial Sequence
  Description of Artificial Sequence Synthetic Peptide
AKSPMDII

SEQ ID NO: 38 - protein, 8 aa, Artificial Sequence
  Description of Artificial Sequence Synthetic Peptide
AKGSMRLI

SEQ ID NO: 39 - protein, 8 aa, Artificial Sequence
  Description of Artificial Sequence Synthetic Peptide
AKLTMSKI

SEQ ID NO: 40 - protein, 8 aa, Artificial Sequence
  Description of Artificial Sequence Synthetic Peptide
AKAWMDMI

SEQ ID NO: 41 - protein, 8 aa, Artificial Sequence
  Description of Artificial Sequence Synthetic Peptide
LGNYLRGI

SEQ ID NO: 42 - protein, 8 aa, Artificial Sequence
  Description of Artificial Sequence Synthetic Peptide
LGSVLSFI

SEQ ID NO: 43 - protein, 8 aa, Artificial Sequence
  Description of Artificial Sequence Synthetic Peptide
LGGALDPI

SEQ ID NO: 44 - protein, 8 aa, Artificial Sequence
  Description of Artificial Sequence Synthetic Peptide
LGLRLRSI

SEQ ID NO: 45 - protein, 8 aa, Artificial Sequence
  Description of Artificial Sequence Synthetic Peptide
LGANLSTI

SEQ ID NO: 46 - protein, 8 aa, Artificial Sequence
  Description of Artificial Sequence Synthetic Peptide
LGNDMDWI

SEQ ID NO: 47 - protein, 8 aa, Artificial Sequence
  Description of Artificial Sequence Synthetic Peptide
LGSCMRYI

SEQ ID NO: 48 - protein, 8 aa, Artificial Sequence
  Description of Artificial Sequence Synthetic Peptide
LGGEMSVI

SEQ ID NO: 49 - protein, 8 aa, Artificial Sequence
  Description of Artificial Sequence Synthetic Peptide
LGLQMDAI

SEQ ID NO: 50 - protein, 8 aa, Artificial Sequence
  Description of Artificial Sequence Synthetic Peptide
LGAGMRRI

SEQ ID NO: 51 - protein, 8 aa, Artificial Sequence
  Description of Artificial Sequence Synthetic Peptide
LKNHLSNI

SEQ ID NO: 52 - protein, 8 aa, Artificial Sequence
  Description of Artificial Sequence Synthetic Peptide
LKSILDDI

SEQ ID NO: 53 - protein, 8 aa, Artificial Sequence
  Description of Artificial Sequence Synthetic Peptide
LKGLLRCI

SEQ ID NO: 54 - protein, 8 aa, Artificial Sequence
  Description of Artificial Sequence Synthetic Peptide
LKLKLSEI

SEQ ID NO: 55 - protein, 8 aa, Artificial Sequence
  Description of Artificial Sequence Synthetic Peptide
LKAMLDQI

SEQ ID NO: 56 - protein, 8 aa, Artificial Sequence
  Description of Artificial Sequence Synthetic Peptide
LKNGMRGI

SEQ ID NO: 57 - protein, 8 aa, Artificial Sequence
  Description of Artificial Sequence Synthetic Peptide
LKSFMSHI

SEQ ID NO: 58 - protein, 8 aa, Artificial Sequence
  Description of Artificial Sequence Synthetic Peptide
LKGPMDII

SEQ ID NO: 59 - protein, 8 aa, Artificial Sequence
  Description of Artificial Sequence Synthetic Peptide
LKLSMRLL

SEQ ID NO: 60 - protein, 8 aa, Artificial Sequence
  Description of Artificial Sequence Synthetic Peptide
LKATMSKL

SEQ ID NO: 61 - protein, 8 aa, Artificial Sequence
  Description of Artificial Sequence Synthetic Peptide
FGNWLSML

SEQ ID NO: 62 - protein, 8 aa, Artificial Sequence
  Description of Artificial Sequence Synthetic Peptide
FGSYLDGL

SEQ ID NO: 63 - protein, 8 aa, Artificial Sequence
  Description of Artificial Sequence Synthetic Peptide
FGGVLRFL

SEQ ID NO: 64 - protein, 8 aa, Artificial Sequence
  Description of Artificial Sequence Synthetic Peptide
FGLALDPL

SEQ ID NO: 65 - protein, 8 aa, Artificial Sequence
  Description of Artificial Sequence Synthetic Peptide
FGARLRSL

SEQ ID NO: 66 - protein, 8 aa, Artificial Sequence
  Description of Artificial Sequence Synthetic Peptide
FGNNMSTL

SEQ ID NO: 67 - protein, 8 aa, Artificial Sequence
  Description of Artificial Sequence Synthetic Peptide
FGSDMDWL

SEQ ID NO: 68 - protein, 8 aa, Artificial Sequence
  Description of Artificial Sequence Synthetic Peptide
FGGRMDRI

SEQ ID NO: 69 - protein, 8 aa, Artificial Sequence
  Description of Artificial Sequence Synthetic Peptide
FGLEMSVL

SEQ ID NO: 70 - protein, 8 aa, Artificial Sequence
  Description of Artificial Sequence Synthetic Peptide
FGAQMDAL

SEQ ID NO: 71 - protein, 8 aa, Artificial Sequence
  Description of Artificial Sequence Synthetic Peptide
FKNGLRRL

SEQ ID NO: 72 - protein, 8 aa, Artificial Sequence
  Description of Artificial Sequence Synthetic Peptide
FKSHLSNL

SEQ ID NO: 73 - protein, 8 aa, Artificial Sequence
  Description of Artificial Sequence Synthetic Peptide
FKGILDDL

SEQ ID NO: 74 - protein, 8 aa, Artificial Sequence
  Description of Artificial Sequence Synthetic Peptide
FKLLLRCL

SEQ ID NO: 75 - protein, 8 aa, Artificial Sequence
  Description of Artificial Sequence Synthetic Peptide
FKAKLSEL

SEQ ID NO: 76 - protein, 8 aa, Artificial Sequence
  Description of Artificial Sequence Synthetic Peptide
FKNMMDQL

SEQ ID NO: 77 - protein, 8 aa, Artificial Sequence
  Description of Artificial Sequence Synthetic Peptide
FKSGMRGL

SEQ ID NO: 78 - protein, 8 aa, Artificial Sequence
  Description of Artificial Sequence Synthetic Peptide
FKGFMSHL

SEQ ID NO: 79 - protein, 8 aa, Artificial Sequence
  Description of Artificial Sequence Synthetic Peptide
FKLPMDIL

SEQ ID NO: 80 - protein, 8 aa, Artificial Sequence
  Description of Artificial Sequence Synthetic Peptide
FKASMRLL

SEQ ID NO: 81 - protein, 8 aa, Artificial Sequence
  Description of Artificial Sequence Synthetic Peptide
AGNTLSKL

SEQ ID NO: 82 - protein, 8 aa, Artificial Sequence
  Description of Artificial Sequence Synthetic Peptide
AGSWLDML

SEQ ID NO: 83 - protein, 8 aa, Artificial Sequence
  Description of Artificial Sequence Synthetic Peptide
AGGYLRGL

SEQ ID NO: 84 - protein, 8 aa, Artificial Sequence
  Description of Artificial Sequence Synthetic Peptide
AGLVLSFL

SEQ ID NO: 85 - protein, 8 aa, Artificial Sequence
  Description of Artificial Sequence Synthetic Peptide
AGAALSPL

SEQ ID NO: 86 - protein, 8 aa, Artificial Sequence
  Description of Artificial Sequence Synthetic Peptide
AGNRMRSL

SEQ ID NO: 87 - protein, 8 aa, Artificial Sequence
  Description of Artificial Sequence Synthetic Peptide
AGSNMSTL

SEQ ID NO: 88 - protein, 8 aa, Artificial Sequence
  Description of Artificial Sequence Synthetic Peptide
AGGDMDWL

SEQ ID NO: 89 - protein, 8 aa, Artificial Sequence
  Description of Artificial Sequence Synthetic Peptide
AGLCMRYL

SEQ ID NO: 90 - protein, 8 aa, Artificial Sequence
  Description of Artificial Sequence Synthetic Peptide
AGAEMSVL

SEQ ID NO: 91 - protein, 8 aa, Artificial Sequence
  Description of Artificial Sequence Synthetic Peptide
AKNQLDAL

SEQ ID NO: 92 - protein, 8 aa, Artificial Sequence
  Description of Artificial Sequence Synthetic Peptide
AKSGLRRL

SEQ ID NO: 93 - protein, 8 aa, Artificial Sequence
  Description of Artificial Sequence Synthetic Peptide
AKGHLSNL

SEQ ID NO: 94 - protein, 8 aa, Artificial Sequence
  Description of Artificial Sequence Synthetic Peptide
AKLILDDL

SEQ ID NO: 95 - protein, 8 aa, Artificial Sequence
  Description of Artificial Sequence Synthetic Peptide
AKALLRCL

SEQ ID NO: 96 - protein, 8 aa, Artificial Sequence
  Description of Artificial Sequence Synthetic Peptide
AKNKMSEL

SEQ ID NO: 97 - protein, 8 aa, Artificial Sequence
  Description of Artificial Sequence Synthetic Peptide
AKSMMDQL

SEQ ID NO: 98 - protein, 8 aa, Artificial Sequence
  Description of Artificial Sequence Synthetic Peptide
AKGGMRGL

SEQ ID NO: 99 - protein, 8 aa, Artificial Sequence
  Description of Artificial Sequence Synthetic Peptide
AKLFMSHL

SEQ ID NO: 100 - protein, 8 aa, Artificial Sequence
  Description of Artificial Sequence Synthetic Peptide
AKAPMDIL

SEQ ID NO: 101 - protein, 8 aa, Artificial Sequence
  Description of Artificial Sequence Synthetic Peptide
LGNSLRLL

SEQ ID NO: 102 - protein, 8 aa, Artificial Sequence
  Description of Artificial Sequence Synthetic Peptide
LGSTLSKL

SEQ ID NO: 103 - protein, 8 aa, Artificial Sequence
  Description of Artificial Sequence Synthetic Peptide
LGGWLDML

SEQ ID NO: 104 - protein, 8 aa, Artificial Sequence
  Description of Artificial Sequence Synthetic Peptide
LGLYLRGL

SEQ ID NO: 105 - protein, 8 aa, Artificial Sequence
  Description of Artificial Sequence Synthetic Peptide
LGAVLSFL

SEQ ID NO: 106 - protein, 8 aa, Artificial Sequence
  Description of Artificial Sequence Synthetic Peptide
LGNAMDPL

SEQ ID NO: 107 - protein, 8 aa, Artificial Sequence
  Description of Artificial Sequence Synthetic Peptide
LGSRMRSL

SEQ ID NO: 108 - protein, 8 aa, Artificial Sequence
  Description of Artificial Sequence Synthetic Peptide
LGGNMSTL

SEQ ID NO: 109 - protein, 8 aa, Artificial Sequence
  Description of Artificial Sequence Synthetic Peptide
LGLDMDWL

SEQ ID NO: 110 - protein, 8 aa, Artificial Sequence
  Description of Artificial Sequence Synthetic Peptide
LGACMRYL

SEQ ID NO: 111 - protein, 8 aa, Artificial Sequence
  Description of Artificial Sequence Synthetic Peptide
LKNELSVL

SEQ ID NO: 112 - protein, 8 aa, Artificial Sequence
  Description of Artificial Sequence Synthetic Peptide
LKSQLDAL

SEQ ID NO: 113 - protein, 8 aa, Artificial Sequence
  Description of Artificial Sequence Synthetic Peptide
LKGGLRRL

SEQ ID NO: 114 - protein, 8 aa, Artificial Sequence
  Description of Artificial Sequence Synthetic Peptide
LKLHLSNL

SEQ ID NO: 115 - protein, 8 aa, Artificial Sequence
  Description of Artificial Sequence Synthetic Peptide
LKAILDDL

SEQ ID NO: 116 - protein, 8 aa, Artificial Sequence
  Description of Artificial Sequence Synthetic Peptide
LKNLMRCL

SEQ ID NO: 117 - protein, 8 aa, Artificial Sequence
  Description of Artificial Sequence Synthetic Peptide
LKSKMSEL

SEQ ID NO: 118 - protein, 8 aa, Artificial Sequence
  Description of Artificial Sequence Synthetic Peptide
LKGMMDQL

SEQ ID NO: 119 - protein, 8 aa, Artificial Sequence
  Description of Artificial Sequence Synthetic Peptide
LKLGMRGL

SEQ ID NO: 120 - protein, 8 aa, Artificial Sequence
  Description of Artificial Sequence Synthetic Peptide
LKAFMSHL

SEQ ID NO: 121 - protein, 15 aa, Artificial Sequence
  Description of Artificial Sequence Synthetic Peptide
PNEEAGAALSPLPEV

SEQ ID NO: 122 - protein, 15 aa, Artificial Sequence
  Description of Artificial Sequence Synthetic Peptide
RSSCAGAALSPLGAC

SEQ ID NO: 123 - protein, 8 aa, Artificial Sequence
  Description of Artificial Sequence Synthetic Peptide
AGAALSPL

SEQ ID NO: 124 - protein, 15 aa, Artificial Sequence
  Description of Artificial Sequence Synthetic Peptide
RSSCFGGRIDRIGAC

SEQ ID NO: 125 - protein, 37 aa, Artificial Sequence
  Description of Artificial Sequence Synthetic Peptide
EVVPPQVLSEPNEEAGAALSPLPEVPPWTGEVSPAQR

SEQ ID NO: 126 - protein, 28 aa, Artificial Sequence
  Description of Artificial Sequence Synthetic Peptide
SLRRSSCFGGRMDRIGAQSGLGCNSFRY

SEQ ID NO: 127 - protein, 8 aa, Artificial Sequence
  Description of Artificial Sequence Synthetic Peptide
FGLKLDRI

SEQ ID NO: 128 - protein, 8 aa, Artificial Sequence
  Description of Artificial Sequence Synthetic Peptide
FKNLLDHL

SEQ ID NO: 129 - protein, 8 aa, Artificial Sequence
  Description of Artificial Sequence Synthetic Peptide
LKSKLRAL

SEQ ID NO: 130 - protein, 8 aa, Artificial Sequence
  Description of Artificial Sequence Synthetic Peptide
FGKRMDRI

SEQ ID NO: 131 - protein, 8 aa, Artificial Sequence
  Description of Artificial Sequence Synthetic Peptide
XXXXXXXX

SEQ ID NO: 132 - protein, 15 aa, Artificial Sequence
  Description of Artificial Sequence Synthetic Peptide
RSSCAGKALSPLGAC